(19) United States
(12) Patent Application Publication
(10) Pub. No.: US 2025/0143992 A1
(43) Pub. Date: May 8, 2025

(54) VAT PLUS OXIDATIVE COLORS

(71) Applicant: WELLA GERMANY GMBH, Darmstadt (DE)

(72) Inventors: Markus SPECKBACHER, Darmstadt (DE); Ingo WEBER, Darmstadt (DE); Petra BRAUN, Darmstadt (DE); Sonja TELSER, Darmstadt (DE); Tatjana SCHAEFER, Darmstadt (DE); Olga PRYADILOVA, Darmstadt (DE)

(21) Appl. No.: 18/837,463

(22) PCT Filed: Feb. 10, 2023
(86) PCT No.: PCT/EP2023/053358

§ 371 Date: Aug. 9, 2024

(30) Foreign Application Priority Data
Feb. 10, 2022 (EP) 22156189.7

(51) Int. Cl. A61K 8/49; A61Q 5/10

(52) U.S. Cl. CPC A61K 8/494; A61Q 5/10; A61K 2800/4322

(57) ABSTRACT

Methods and compositions for coloring keratinous substrates, such as mammalian hair and in particular human hair, use oxidative dye precursors comprising pyrazole primaries and couplers, and solubilized vat dyes. The methods and compositions improve the wash fastness of hair color produced by oxidative dyes, wherein one or more of the oxidative dyes are derived from oxidative dye precursors comprising a pyrazole primary and a coupler.

### BACKGROUND OF THE INVENTION

Treatments to mammalian or synthetic keratin fibers are known. Mammalian keratin fibers (natural hair) is structured as a cuticle or outer surface layer, a cortex which is an internal mid layer containing melanin or color bodies and keratin bundles, and a central core termed medulla. Typical dye treatments focus on changes of the cortex. Of particular note are cortex treatments that alter the appearance of the hair, for example by changing the color or reflective properties of hair. This can be achieved through treating hair cortex with a formulation containing dye molecules (so-called direct dyes) which diffuse into or are absorbed through the cuticle of the hair fibers. Over time, the color imparted to the hair is removed during washing, which usually occurs rapidly for the direct dyes. Direct dyes therefore typically lead to a short-term change in hair appearance, with the color imparted to the hair by direct dyes typically lasting for a few washes.

The most common approach used nowadays for producing a permanent or demi-permanent color change of hair involves so-called oxidative dyes, wherein the dye precursors (intermediates called primaries and couplers) diffuse into the cortex and then react to form colored species within the cortex of the hair. Often the oxidative dye products are designed to also lighten the hair, decolorizing some of the melanin of the cortex to enable a wider range of colors to be achieved. Although the color imparted to the hair by oxidative dyes as well is removed during washing, the oxidative dyes may last considerably longer than direct dyes, and indeed removing the color can be hard to achieve completely, even after a considerable number of washes. When oxidative dyes are eventually removed by washing out, the melanin has also been decolorized by bleaching so that it will not return to its original color but to a lighter color, leaving some color traces on the hair.

Another approach for producing a long-lasting color change of hair involves so-called vat dyes. Vat dyes may be brought to a soluble form, for example, by converting vat dye pigments (insoluble in aqueous media) to the corresponding leuco vat dye sulfonic esters via reduction in alkaline medium to give the leuco form, followed by treatment with chloro-sulfonic acid or simply sulfur-trioxide. Leuco vat dye sulfonic esters and methods for producing same are disclosed for example in DE1117242B. Vice versa, the leuco vat dye sulfonic esters can be rapidly cleaved to form the classic leuco form of the corresponding vat dye. Subsequent oxidation precipitates the insoluble vat dye as pigment. The terms “solubilized vat dye”, “leuco vat dye sulfonic ester” and “sulfonic ester of a vat dye” are used interchangeably in the present disclosure.

U.S. Pat. No. 5,364,415A (Lewis et al.) discloses a hair coloring method based on a mild oxidative hair coloring system comprising primary intermediates and couplers, which method contemplates additionally applying one of seven selected solubilized vat dyes to the hair, as color brightening special ingredients, in order to obtain brighter red and yellow colors than obtainable with the common oxidative colorants alone.

DE102004014764A1 (Javet et al.) discloses a hair coloring agent comprising a vat dye and a reducing agent forming an enediol under alkaline conditions, which reducing agent converts the vat dye to the leuco form under alkaline conditions. The reference discloses stabilizing the leuco form by appropriate packaging, a protective gas blanket, the addition of stabilizing and/or protecting substances (for example cationic compounds such as polymers, tensides, metal ions), or esterification. DE102004014763A1 (Javet et al.) discloses a similar hair coloring agent wherein the vat dye is an indigoid vat dye, the composition again comprising the reducing agent forming an enediol under alkaline conditions, further comprising a cationic compound.

US2003/0074745A1 (Vainshalboim et al.) discloses a method and system for dyeing hair without using a chemical oxidant. The dyes used are solubilized indigoid and anthraquinone vat dyes.

US2011/0189116A1 (Gauche et al.) discloses a method of coloring a material, comprising applying to the material a composition comprising a sulfur-containing nucleophile; and applying to the material a composition comprising a dye compound other than a reactive dye.

Although solubilized vat dyes are versatile to use in hair coloring, and provide for properties in terms of light and wash fastness comparable to those of oxidative dyes, they have not gained the commercial importance of oxidative dye systems in hair coloring.

With the advent of pyrazole primaries in the mid 1990's, new color spaces for fashion hair colors became accessible, and the pyrazole primaries revolutionized hair coloring in particular for the red shades such as the shades known today as “vibrant reds”. There is a wide range of literature on the pyrazole primaries, and their impact on hair colors in orange-to-red color shades, es exemplified by the following patents and patent applications.

EP0375977A1 discloses oxidative dyes for coloring hair on basis of precursors such as 3,5-diaminopyrazoles and 4,5-diaminopyrazoles for producing brilliant hair colors with high color depth.

DE4234885A1 discloses a process for producing 4,5-diaminopyrazoles for use as dye precursors in oxidative hair coloring.

U.S. Pat. No. 5,380,340A discloses pyrazole derivatives such as 7-amino-2,3-dihydro-1H-imidazo[1,2-b]pyrazole, 3-nitro-4,5,6,7-tetrahydropyrazolo-[1,5-a]pyrimidine or 3-amino-4,5,6,7-tetrahydropyrazolo-[1,5-a]pyrimidine for oxidative hair dyeing.

WO02/051372A2 discloses compositions for oxidation dyeing of keratinous fibres comprising at least a 4,5- or 3,4-diaminopyrazole or a triaminopyrazole and at least a particular cellulose compound.

EP1488783B1 discloses a dyeing composition comprising 4,5-diamino-1-(beta-hydroxyethyl)-1H-pyrazole or 4,5-diamino-1-(beta-methoxyethyl)-1H-pyrazole as oxidation base and 2,6-dihydroxy-3,4-dimethylpyridine as coupling agent.

EP2628731A1 discloses a composition for the oxidative dyeing of keratin fibers, in particular human keratin fibers, comprising as a primary intermediate 1-hexyl-1H-pyrazole-4,5-diamine hemisulfate.

One major drawback of the known oxidation-based technologies in this area is that the overall wash fastness of the coloration experience is not consistent over all shades including natural shades, fashion shades (e.g. vibrant reds etc.) and blond tones. Even under gentle oxidative conditions at moderate alkaline pH (such as pH=9), the wash fastness of various shades is not consistent, namely fashionable red tones always show greater tendencies to be washed out at a higher rate versus darker natural tones.

Such an insufficient wash fastness of the hair colors produced, in particular of the red fashion shades, i.e. the shades produced using diaminopyrazoles as primaries, is well known in the technical field for more than a decade. For example, US2020/019728A1 is directed to an agent for the oxidative dyeing of keratinic fibers, which agent comprises in a cosmetic carrier a 3-alkyl-4,5-diaminopyrazole, iminodisuccinic acid or a salt thereof, and at least one oxidizing agent other than atmospheric oxygen. The reference specifically acknowledges that “many attempts have been made to optimize the color intensity and the fastness properties of fashion shades. However, despite the large number of optimization experiments already carried out, there is still need for improvement with regard to the fastness properties of the dyeing of oxidatively dyed keratin fibers, in particular when they are to be dyed in a fashion shade in the red region”.

There is therefore a need for hair coloring compositions and methods that address, the foregoing drawbacks of known technologies. It has now surprisingly been found a solution that all drawbacks of traditional oxidative hair colorants as stated above can be addressed and conclusively solved at an exceptional high level of professional claims by the invention presented herein.

Solubilized vat dyes, in particular from the broader family of Indanthrene dyes (e.g. featuring anthrachinones, acridine, perylene, naphthaline and violanthrone backbones), applied in combination with known oxidative primaries and couplers in mild oxidative medium (e.g. hydrogen peroxide levels in the range of 1-12%) have demonstrated to improve the wash fastness of the classic oxidative hair colors obtained with pyrazole primaries and couplers alone, or pyrazole primaries and couplers in combination with direct dyes.

The achieved color results according to the inventive concept show significantly improved color lastingness with extraordinary wash and lightfastness properties, starting from mild oxidative conditions at hydrogen peroxide levels<2% up to higher hydrogen peroxide levels such as 9% or 12%. This concept provides full flexibility and artistic freedom for stylists creating any possible shade with significantly improved lastingness across all shades (natural, fashion and blond shades).

### SUMMARY

The present invention addresses the well-known wash fastness deficiencies of oxidative dyeing systems using oxidative dye precursors comprising pyrazole primaries and couplers. According to one aspect, subject matter of the present invention is a method for coloring a keratinous substrate, which method comprises:
a1 applying a composition A to said keratinous substrate, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof,
a2 applying a composition 8 to said keratinous substrate, said composition 8 comprising an aqueous medium and a solubilized vat dye dissolved in said aqueous medium,
b subsequent to step a2, applying a composition C to said keratinous substrate, said composition C comprising an oxidizing agent.

According to another aspect, further subject matter of the present invention is a ready-to-use hair coloring formulation. The hair coloring formulation is obtained by mixing a composition A with a composition B, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, said composition B comprising an aqueous medium and a solubilized vat dye dissolved in said aqueous medium.

According to still another aspect, further subject matter of the present invention is a kit for coloring hair. The kit comprises in separate compartments:
a compartment A comprising a composition A, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, and
a compartment B comprising a composition 8, said composition B comprising an aqueous medium and a solubilized vat dye dissolved in said aqueous medium.

According to yet another aspect, further subject matter of the present invention is a hair coloring component. The hair coloring component may have a creamy, gel-like consistency. Such gel-like hair coloring component may comprise one or more solubilized vat dyes, each in a concentration of 0.50-2.0 wt %, 10.0-30.0 wt % of one or more saturated or unsaturated fatty acids, and 50-89.50 wt % water, wherein wt % each is based on the weight of the component. Alternatively, the hair coloring component may have the consistency of a shampoo, having a composition. Such shampoo-like hair coloring component may comprise one or more solubilized vat dyes, each in a concentration of 0.50-2.0 wt %, 2.0-40.0 wt % anionic surfactant, 2.0-20.0 wt % amphoteric surfactant, 0-10.0 wt % of an optional humectant, and 50.0-95.50 wt % water, wherein wt % each is based on the weight of the component.

Further subject matter of the present invention is the use of a hair coloring component according to the present invention for improving the wash fastness of hair color produced by oxidative dyes, wherein one or more of the oxidative dyes are derived from oxidative dye precursors comprising a pyrazole primary and a coupler. Still further subject matter of the present invention is the use of a solubilized vat dye for improving the wash fastness of hair color produced by oxidative dyes, wherein one or more of the oxidative dyes are derived from oxidative dye precursors comprising a pyrazole primary and a coupler.

### Definitions

As used in the present disclosure and the appended claims, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term “and/or” in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers. According to embodiments, the term “about” is understood to mean±2 percent of the recited number, numbers or range of numbers.

The term “about 0 wt %” is understood to mean that no substance, compound or material to which zero (0) refers is present, up to a negligible but detectable amount is present, assuming that detectability is on a ppm (parts per million) basis.

Where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being methyl and ethyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “keratinous substrates” as used herein means any natural or synthetic substrate made of or comprising a keratin. In particular, the term “keratinous substrates” encompasses natural and synthetic keratin fibers. Preferred keratinous substrates are natural keratin fibers. Natural keratin fibers include those from mammals and/or on mammals including human, primate, ruminant, camelid, equine, rodent and neovison including but not limited to cow, sheep, deer, goat, buffalo, lama, alpaca, camel, guanaco, vicuna, horse, antelope, moose, elk, rat, mouse, beaver, rabbit, mink, monkey, ape and similar species. Natural keratin fibers may include hair, fur or nails. Particularly preferred keratinous substrates in the present disclosure are mammalian keratin fibers, in particular human hair, and more particularly human scalp hair. The term “keratin fibers” is used interchangeably in this document with the terms “hair” or “hair strands” unless the context dictates otherwise.

As used herein, the term “optionally” means that the corresponding substituent or thing may or may not be present. It includes both possibilities.

The term “water-soluble” as used herein refers to a solubility at 25° C. of at least 1.0 wt %, in particular at least 5.0 wt %, for example at least 10.0 wt % such as at least 25.0 wt %.

The term “composition” as used herein denotes a mixture or blend comprising at least two components. Typical compositions comprise at least one functional component (such as, for example, a leuco vat dye sulfonic ester, or a pH adjuster, or an oxidizing or reducing agent) and at least one solvent component (such as water or an alcohol). A composition may be present in any suitable physical form, including for example liquids of varying viscosities, gels, foams, creams, and the like. The term “formulation” as used herein denotes the ready-to-use composition unless the context dictates otherwise. “Ready-to-use” denotes the form in which the composition//formulation is actually applied to a keratinous substrate such as mammalian hair, in particular human (scalp) hair.

The term “alkalizer” as used herein denotes any compound suitable for adjusting the pH of a composition in the alkaline range. Typical alkalizers comprise bases such as NaOH, ammonia, urea, pyridine, aminomethylpropanol (AMP), monoethanolamine, 2-amino-1-propanol, DBU (diazabicyclo-undecane). Pyridine and DBU may give create safety issues and for that reason are less preferred.

The term “essentially free” of a particular component denotes an amount of less than 1.0 wt % of the respective component in an entity such as a composition, based on the total weight of the entity. In particular, the term “essentially free” denotes an amount of less than 0.5 wt %, for example less than 0.1 wt %, such as 0.01 wt % of the respective component in the entity. The term “free” of a particular component denotes an amount below the detection limit, assuming that detectability is on a ppm (parts per million) basis.

The term “oxidative dye percursors” or simply “dye precursors” or “color precursors” refers to compounds that may be used in the composition(s) of the present invention to act as primary intermediates, couplers, or both, in order to provide color to keratinous substrates.

The term “dye” as used herein refers to reaction products of the “oxidative dye percursors”, and to the finally obtained oxidized and insoluble form of the vat dyes. Although the leuco form of the vat dyes may be referred to as “dye” herein, such leuco forms usually are uncolored or have an insufficient color depth. The term “dye” as used herein implies at least one absorption maximum of at least 0.001 AU/g/l, preferably at least 0.01 AU/g/l (absorption units per gram per liter) in the visible range (400-700 nm). Further the term “dye” as used herein excludes direct dyes. Direct dyes exhibit a low wash fastness, would become oxidized in the conditions used in the present invention, and accordingly are excluded from the present invention. Finally, the term “dye” as used herein excludes inorganic pigments.

### DETAILED DESCRIPTION

Subject matter of the present invention is a novel and highly versatile concept for the dyeing of keratinous fibers, in particular mammalian hair and more particularly human hair. The concept of the present invention combines oxidative hair coloring technology using pyrazole primaries for fashion shades with vat dye technology. The combination of the two technologies unexpectedly achieves an improved lastingness or wash fastness of the color imparted by the oxidative coloring system, together with the known excellent lastingness or wash fastness of the color imparted by the vat dyes.

In short words, the present invention involves compositions A and B, which are applied to the keratinous substrate such as human hair subsequently, simultaneously or in mix form. Composition A provides for oxidative hair coloring technology, comprising dye precursors, which oxidative dye precursors comprise one or more pyrazole primaries and one or more couplers. Composition B provides for vat dye technology, comprising one or more solubilized vat dyes.

### 1 Composition a and the Oxidative Dye Precursors

The present invention uses a composition (denoted composition A) involving oxidative dye chemistry, using oxidative dye precursors comprising a pyrazole primary (also known as primary intermediate or developer) and a coupler (also known as secondary intermediate). One or more couplers may be used with one or more primaries in order to obtain different shades. The primaries used may comprise-in addition to the pyrazole primaries-one or more traditional primaries. The shades contemplated by the present invention include in particular so-called fashion shades such as shades in the color space including orange, reddish-orange, copper, mahogany red, mahogany brownish, and vibrant reds.

The oxidative dye precursors suitable for use herein, in so far as they are bases, may be used as free bases or in the form of any cosmetically acceptable salts obtained with the corresponding organic or inorganic acids, such as hydrochloric, hydrobromic, citric, acetic, lactic, succinic, tartaric, or sulfonic acids, or, in so far as they have aromatic hydroxyl groups, in the form of any cosmetically acceptable salts obtained with the corresponding bases, such as alkali phenolates.

Oxidative dye precursors are known in the art, and include aromatic diamines, aminophenols, aromatic diols and their derivatives (a representative but not exhaustive list of oxidation dye precursors can be found in Sagarin, “Cosmetic Science and Technology, Interscience, Special Edn. Vol. 2 pages 308 to 310). Suitable oxidative dye precursors are also disclosed in the Canadian Patent Application No. CA 2 576 189 A1-in particular, from Table 1 dye combinations No. 1 to 2394, which span pages 49 to 238, are incorporated herein by reference. It is to be understood that the one or more primary intermediates and the one or more couplers (collectively known as oxidative dye precursors) detailed below are only by way of example and are not intended to limit the present invention. The one or more primary intermediates and the one or more couplers may be used in the form of any cosmetically acceptable salts, for example sulfate salts.

Composition A comprises a pyrazole primary and a coupler, wherein the pyrazole primary comprises a 4,5-diaminopyrazole, an aminopyrazolopyridine, a ring-condensed 4,5-diaminopyrazolone, or a combination thereof.

According to preferred embodiments, composition A comprises a 4,5-diaminopyrazole according to Formula (1):

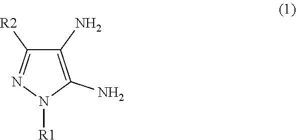

wherein R1 is linear or branched C1-C6 (hydroxy)alkyl and R2 is H or linear or branched C1-C6 (hydroxy)alkyl. Examples of 4,5-diaminopyrazoles according to Formula (1) encompass 1-hydroxyethyl-4,5-diaminopyrazole, 4,5-diamino-1-methylpyrazole, 4,5-diamino-1-ethylpyrazole, 4,5-diamino-1-(iso) propylpyrazole, 4,5-diamino-1-butylpyrazole, 4,5-diamino-1-pentylpyrazole, 4,5-diamino-3-methylpyrazol, and 4,5-diamino-1-hexylpyrazole.

According to particular embodiments, the pyrazole primary in composition A comprises a 4,5-diaminopyrazole selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.

According to embodiments, composition A comprises an aminopyrazolopyridine according to Formula (2):

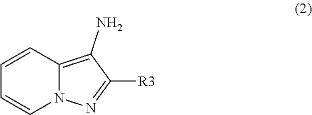

wherein R3 is (2-hydroxyethyl)oxy or 4,4-dimethylpiperazinium, or a combination thereof.

According to embodiments, composition A comprises a ring-condensed 4,5-diaminopyrazolone according to Formula (3):

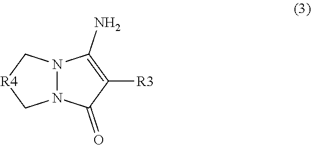

wherein R4 is methylene, ethylene, propylene, or a combination thereof.

Examples of pyrazole primaries suitable for the present invention but not falling under Formula (1) encompass 4,5-diamino-1-heptylpyrazole, 4,5-diamino-1-benzylpyrazole, 2,3-diamino-6,7-dihydropyrazolo[1,2-a]pyrazol-1 (5H)-one.

According to preferred embodiments, composition A comprises a pyrazole primary selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, 2,3-diaminodihydropyrazolopyrazolone (CAS #857035-94-0), dimethylpiperaziniumaminopyrazolopyridine (CAS #1256553-33-9), 3-Amino-2-((2-hydroxyethyl)oxy)-pyrazolo[1,5-a]pyridin (CAS #1079221-49-0), or a combination thereof. According to yet more particular embodiments, composition A comprises a pyrazole primary selected from 4,5-diamino-1-methylpyrazol, 4,5-diamino-1-ethylpyrazol, 4,5-diamino-1-hexylpyrazol, 4,5-diamino-1-hydroxyethylpyrazol, or a combination thereof.

Examples of conventional primaries which are not pyrazole primaries, and which may be present in composition A in addition to the one or more pyrazole primaries encompass toluene-2,5-diamine, p-phenylenediamine, N-phenyl-p-phenylenediamine, N,N-bis(2-hydroxyethyl)-p-phenylenediamine, 2-hydroxyethyl-p-phenylenediamine, hydroxypropyl-bis-(N-hydroxyethyl-p-phenylenediamine), 3-hydroxypropyl-p-phenylenediamine, 2-methyl-p-phenylenediamine, 2-methoxymethyl-p-phenylenediamine, 2-(1,2-dihydroxyethyl)-p-phenylenediamine, 2,2′-(2-(4-aminophenylamino) ethylazanediyl) diethanol, 2-(2,5-diamino-4-methoxyphenyl) propane-1,3-diol, 2-(7-amino-2H-benzo[b][1,4]oxazin-4 (3H)-yl) ethanol, 2-chloro-p-phenylenediamine, p-aminophenol, p-(methylamino) phenol, 4-amino-m-cresol, 6-amino-m-cresol, 5-ethyl-o-aminophenol, 2-methoxy-p-phenylenediamine, 2,2′-methylenebis-4-aminophenol, 2,4,5,6-tetraminopyrimidine, 2,5,6-triamino-4-pyrimidinol, and combinations thereof.

According to particular embodiments, the conventional primary intermediates optionally present in composition A may comprise N,N-bis(2-hydroxyethyl)-p-phenylenediamine, 4-aminophenol, p-phenylenediamine, 3-hydroxypropyl-p-phenylenediamine, 2-methyl-p-phenylenediamine, 2-methoxymethyl-p-phenylenediamine, or a combination thereof.

The one or more couplers may be a compound comprising at least one phenyl ring substituted with at least one hydroxyl group. The one or more couplers may be selected from the group consisting of resorcinol, 4-chlororesorcinol, 2-chlororesorcinol, 2-methylresorcinol, 4,6-dichlorobenzene-1,3-diol, 2,4-dimethylbenzene-1,3-diol, m-aminophenol, 4-amino-2-hydroxytoluene, 2-methyl-5-hydroxyethylaminophenol, 3-amino-2,6-dimethylphenol, 3-amino-2,4-dichlorophenol, 5-amino-6-chloro-o-cresol, 5-amino-4-chloro-o-cresol, 6-hydroxybenzomorpholine, 2-amino-5-ethylphenol, 2-amino-5-phenylphenol, 2-amino-5-methylphenol, 2-amino-6-methylphenol, 2-amino-5-ethoxyphenol, 5-methyl-2-(methylamino) phenol, 2,4-diaminophenoxyethanol, 2-amino-4-hydroxyethylaminoanisole, 1,3-bis-(2,4-diaminophenoxy)-propane, 2,2′-(2-methyl-1,3-phenylene)bis(azanediyl) diethanol, benzene-1,3-diamine, 2,2′-(4,6-diamino-1,3-phenylene)bis(oxy) diethanol, 3-(pyrrolidin-1-yl) aniline, 1-(3-(dimethylamino)phenyl) urea, 1-(3-aminophenyl) urea, 1-naphthol, 2-methyl-1-naphthol, 1,5-naphthalenediol, 2,7-naphthalenediol, 1-acetoxy-2-methylnaphthalene, 4-chloro-2-methylnaphthalen-1-ol, 4-methoxy-2-methylnaphthalen-1-ol, 2,6-dihydroxy-3,4-dimethylpyridine, 2,6-dimethoxy-3,5-pyridinediamine, 3-amino-2-methylamino-6-methoxypyridine, 2-amino-3-hydroxypyridine, 2,6-diaminopyridine, pyridine-2,6-diol, 5,6-dihydroxyindole, 6-hydroxyindole, 5,6-dihydroxyindoline, 3-methyl-1-phenyl-1H-pyrazol-5 (4H)-one, 1,2,4-trihydroxybenzene, 2-(benzo[d][1,3]dioxol-5-ylamino) ethanol (also known as hydroxyethyl-3,4-methylenedioxyaniline), phenyl methyl pyrazolone, 6-methoxy-2-methylamino-3-aminopyridine, and combinations thereof.

According to particular embodiments, the couplers may comprise resorcinol, 2-methylresorcinol, hydroxyethyl-3,4-methylenedioxyaniline, m-aminophenol, 1-naphthol, 2-amino-4-hydroxyethylaminoanisole, 4-amino-2-hydroxytoluene, 2-methyl-5-hydroxyethylaminophenol, phenyl methyl pyrazolone, 6-hydroxy-benzomorpholine, 6-methoxy-2-methylamino-3-aminopyridine, or a combination thereof.

According to embodiments, composition A comprises each primary in a concentration of 0.005-6.0 wt %, in particular 0.01-3.0 wt %, such as 0.2-2.0 wt %, based on the weight of composition A. According to embodiments, composition A comprises each coupler in a concentration of 0.005-6.0 wt %, in particular 0.01-3.0 wt %, such as 0.2-2.0 wt %, based on the weight of composition A.

Typically, composition A comprises a total amount of oxidative dye precursors, namely the one or more couplers, the one or more pyrazole primaries, and the one or more optional conventional primary intermediates, up to 12.0 wt %, or from 0.1-12.0 wt %, or from 0.2-10.0 wt %, or from 0.5-8.0 wt % by total weight of composition A.

### 2 Composition B and the Solubilized Vat Dyes

Vat dyes typically are used and known for many decades as powerful dyes and pigments for textile dyeing. Especially those of the Indanthrene family show superior properties regarding light fastness, wash fastness, are stable to any oxidizing event and are extremely weather proof. In addition, they do show remarkable melting points>100° C. which indicates the high chemical stability. The only option to chemically modify them are applying reductive processes so they also appear in a soluble and therefore processable form. The typical scheme of transformation options is as follows (scheme 1).

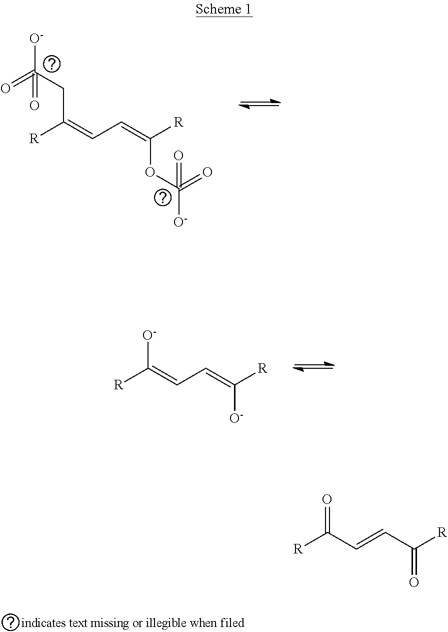

Vat dyes typically require harsh alkaline conditions to get reduced to the corresponding leuco form where they have the ability to penetrate into the fiber as soluble dye. These conditions (pH>13) are not possible to be applied to human hair, hence, the bypass via the labile sulfonic esters is the method of choice. As already mentioned, this reactive and readily soluble form of vat dye pigments has been developed to also color sensitive fibers (e.g. natural fibers such as silk) even their commercial importance is significantly less meanwhile compared to the vat dyes per se.

The sulfonic esters of the vat dyes are perfect candidates as this structural element opens the space to many chromophores which can be modified as such. The ideal chromophore candidate therefore needs to have at least 2 carbonyl groups where reduction and oxidation steps can be applied according to scheme 1 above. In combination with an extended aromatic electronic It-system, typical chromophores are formed, for example of the exceptionally stable Indanthrene family. These solubilized sulfonic ester vat dyes show a totally unexpected ability to penetrate deeply into the cortex of the hair under mild alkaline conditions (pH=9) in presence of swelling promotors such as urea, mono-ethanol amine, 2-amino-1-propanol or ammonia. This observation is not obvious given the size and bulky structure of the chromophores. In comparison with other, also bulky direct dyes (e.g. azo dyes, anthraquinone dyes etc.) with anionic or cationic charges, known to the literature, the observed penetration into the hair cortex is only very moderate to even low and these traditional direct dyes may only be fixed on the outer surface of hair which can be proven via microscopic hair diameter cuts. Hence, this property is remarkable and makes the solubilized vat dyes outstanding from other chromophores in regard of their use as permanent hair colorant. After the penetration phase under alkaline conditions is completed, the cleavage of the labile sulfonic ester group is forced, typically by reducing the pH value to moderately gently acid conditions (pH=3.5) to obtain the pure reduced leuco form which is then rapidly oxidized in presence of air oxygen, hydrogen peroxide and/or bleaching ingredients such as persulfate salts. However, even without an acidifying step, the cleavage of the sulfonic ester group appears to a large extent due to the principal lability of the ester in aqueous media, especially when reductive agents such as citric acid or ascorbic acid are present in a formulation as well. Once the vat dye pigments are precipitated in the cortex of the hair after the re-oxidation is completed, an exceptional long-lasting hair coloration is achieved, caused by the absolute insolubility of the formed pigments.

Hair coloring has reached high attention of toxicologists and is subject to safety clearance by authorities before the compounds can be used in commercial hair color formulations. Hence, a careful selection of appropriate chromophore structures is essential to provide a selection of vat dyes which can be turned into commercial applications. Our intention is to propose a variety of dedicated vat dyes which have a high likelihood to meet all criteria of toxicological pre-requisites. The most important question is if these dyes have the ability to penetrate through skin. Most of all previously published dyes used for hair coloring, regardless if applied under non-oxidative or oxidative conditions, do show a significant tendency to penetrate, simply caused by their water solubility as hydrophilic, anionic or cationic dyes which is a mandatory requirement to process them on hair fibers. Hence, sophisticated and cost intense toxicological evaluations are required in order to obtain safety clearance by authorities. An important improvement versus the state of the art will be, if any skin penetration can be excluded in principal. It is common sense that pigments such as the finally precipitated vat dyes are completely insoluble and not bio available and therefore not of interest for further toxicological evaluations. Hence, the focus here obviously is on the solubilized vat dyes as the intermediate form, which is applied on the hair fiber to process penetration into the cortex of the hair. Hence, only chromophores with large and extended electronic n-systems are considered in order to obtain an acceptable safety evaluation. That excludes all indigoid vat dyes as their molecular weight is regarded to be too low and they will likely penetrate easily through skin. On the other hand, the previously mentioned vat dyes and solubilized vat dyes, respectively, show high molecular weight and the chromophore design is characterized by an even and extended bulky, aromatic polycyclic hydrocarbon surface. This bulky structure shows great hydrophobicity and steric hinderance which significantly decreases the probability of any skin penetration. This is underlined by the relatively moderate LogP values (see a selection of solubilized vat dye LogP values in scheme 2) of the solubilized vat dyes which show a remarkable low hydrophilicity provided their extraordinary good solubility in water caused by the minimum of 2 sulfonic ester groups (see LogWS values).

Scheme 2:

| Sol. Vat dye | LogP | LogWS (intrinsic water solubility) |
|---|---|---|
| Sol vat blue 6 | −0.21 | −2.41 |
| Sol vat orange 3 | 3.08 | −1.83 |
| Sol vat orange 9 | 3.64 | −3.36 |
| Sol vat red 10 | −0.33 | −1.81 |
| Sol vat red 34 | −1.56 | −1.94 |
| Sol vat yellow 1 | 2.65 | −2.48 |
| Sol vat yellow 4 | 2.83 | −1.41 |
| Sol vat violet 1 | 5.15 | −3.19 |
| Sol vat blue 4 | −1.2 | −1.64 |
| Sol vat green 1 | 2.89 | −3.97 |

The dyes are selected from the overall dye family “Indanthrene” dyes which is more a synonym for extra stable dyes with excellent fastness properties rather referring to a dedicated chemical class. Even though, most of the suitable chromophores to be claimed in this invention are selected from dyes based on naphthalene, perylene, anthraquinone and violanthrone backbones. As already mentioned, all do have in common that they have no aromatic amine function but have at least 2 carbonyl groups to be modified to the solubilized sulfonic ester intermediate which is regarded to be the reactive color ingredient for this oxidative hair dyeing system.

These commercial dyes are manufactured according to a standard procedure from commercial vat dye pigments (see DE1117242). The reduction is carried out in alkaline medium using n-methyl acetamide, potassium boronate or sodium dithionite, followed by sulfatization in the presence of sulfur trioxide and di-chloro ethane as solvent. Alternatively, the reaction can be carried out in pyridine as alkalizer and using chlor-sulfonic acid to yield the sulfonic ester. This is exemplary shown for solubilized vat green 1 in scheme 3.

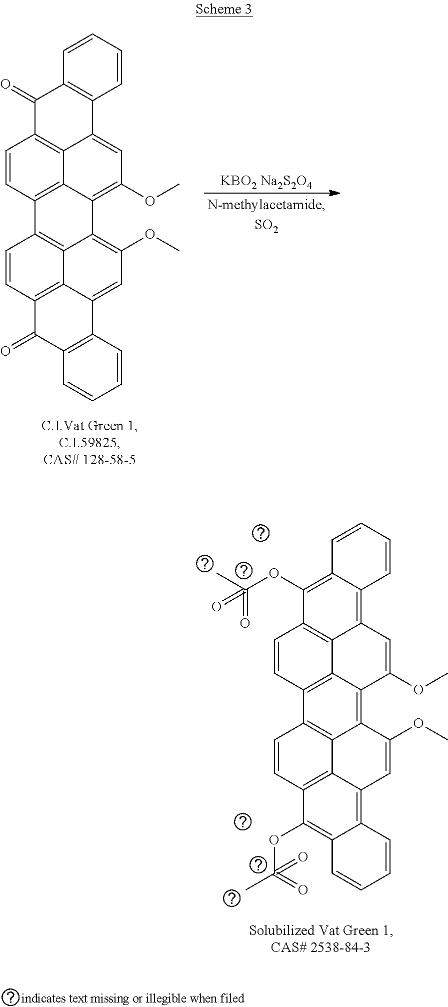

According to the present invention, composition B comprises an aqueous medium and a solubilized vat dye dissolved in said aqueous medium. According to embodiments, composition B comprises two or more solubilized vat dyes. Typically, the solubilized vat dye in composition B is a leuco vat dye sulfonic ester, or a combination of leuco vat dye sulfonic esters.

Preferred solubilized vat dyes for use in the present invention are solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CAS #12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), according to the following structures in scheme 4.

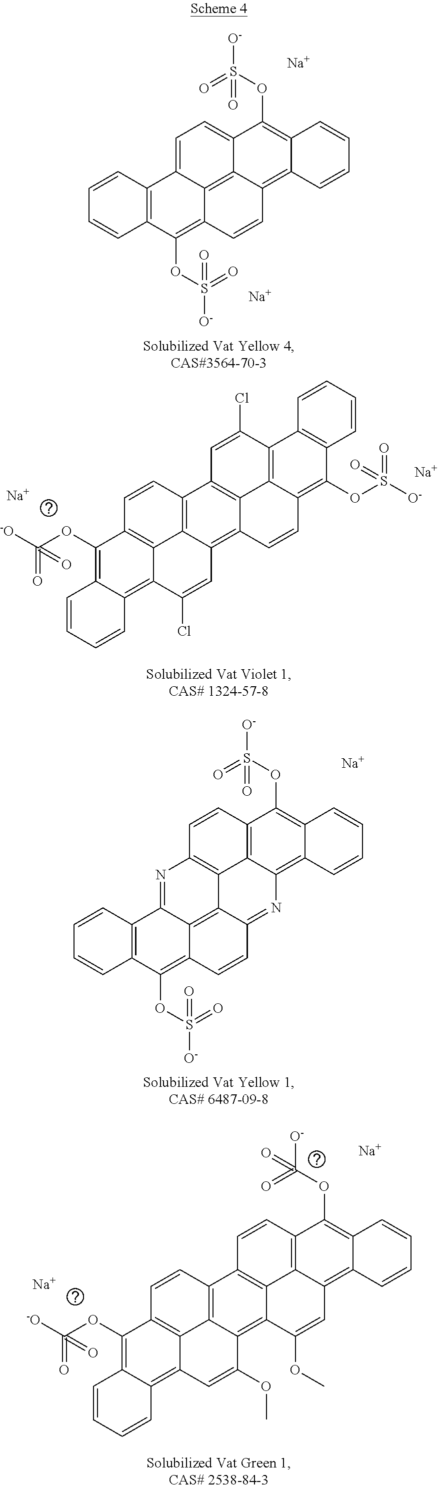

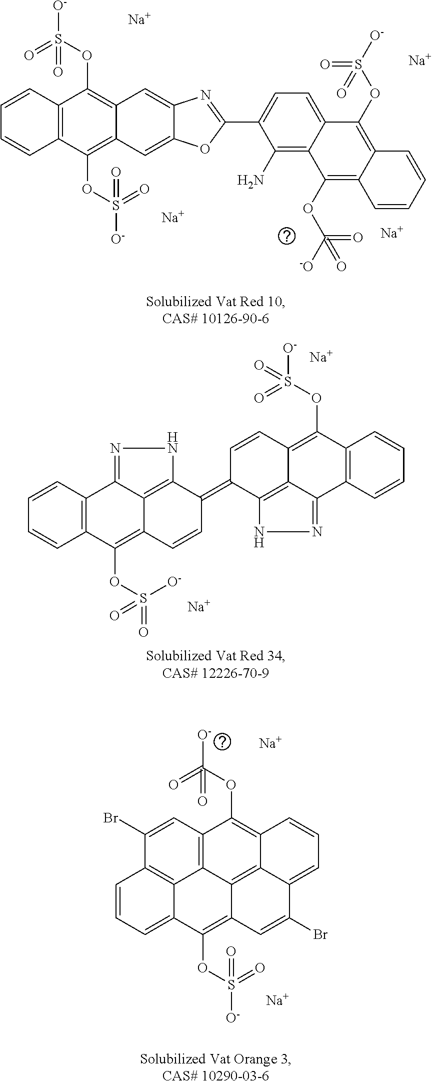

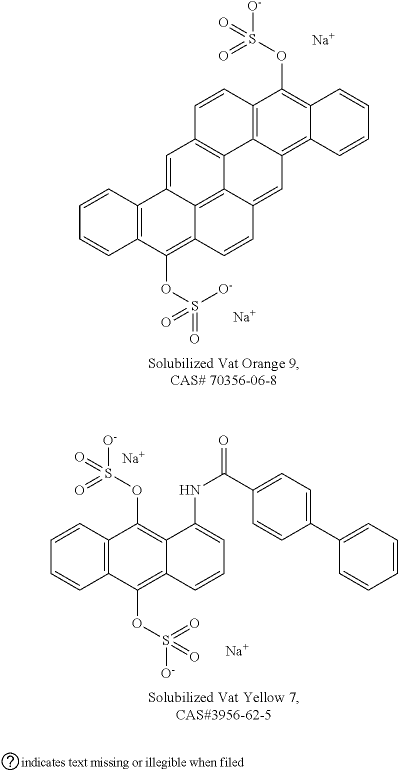 [SMILES: O=S(=O)([O-])Oc1c2ccccc2c2cc3ccc4c(OS(=O)(=O)[O-])c5ccccc5c5cc6ccc1c2c6c3c45 ; [Na+] ; [Na+] ; O=C(Nc1cccc2c(OS(=O)(=O)[O-])c3ccccc3c(OS(=O)(=O)[O-])c12)c1ccc(-c2ccccc2)cc1 ; [Na+] ; [Na+]]

According to particular embodiments, composition B comprises a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.

According to embodiments, composition B comprises each solubilized vat dye in a concentration of 0.50-2.0 wt %, in particular a concentration of 0.6-1.5 wt %, for example a concentration of 0.75-1.0 wt %, based on the weight of composition B. Typically, composition B comprises a total amount of solubilized vat dyes up to 8.0 wt %, or from 0.5-6.0 wt %, or from 1.0-5.0 wt %, or from 1.5-4.0 wt % by total weight of composition B.

### 3 Improvement of Wash Fastness

The present invention discloses a highly versatile hair coloring system based on a combination of oxidative hair coloring technology using pyrazole primaries for fashion shades with vat dye technology. The gist of the invention is not the combination of oxidative hair coloring technology with vat dye technology, however. Instead, the gist of the present invention is the unexpected discovery that the combination of the two technologies results in an improvement of the wash fastness of the color obtained. That is, the known excellent lastingness or wash fastness of the color imparted by the vat dyes is extended to the color imparted by the oxidative dyes. Although it is believed that such improvement principally may be obtained with all color shades (including natural and blond shades), the effect is most pronounced and clearly visible with the fashion shades (including for example the “vibrant reds”). These fashion shades are obtained with oxidative dye systems comprising a pyrazole primary comprising a 4,5-diaminopyrazole, which accordingly forms the starting point of the present invention.

As noted above in the introductory part, U.S. Pat. No. 5,364,415A (Lewis et. al.) proposed introducing solubilized vat dyes as color brightening special ingredients in addition to the common oxidative process applying primaries and couplers to overcome the lack of oxidative colorants to provide bright fashionable shades. However, back in 1994 where this patent dates back, the pyrazoles (e.g. hydroxyethyl-4,5-diamino-pyrazol or hexyl-4,5-diamino-pyrazol), the current global standards as primaries to achieve true intense and vibrant red shades, where not yet in use and not subject to this publication. Hence, Lewis et al. focus on color brightening effects achieved by mixing solubilized vat dyes with oxidative primaries and couplers to achieve brighter shades. The pyrazole blockbusters for creating today's fashion shades were still not available at the time U.S. Pat. No. 5,364,415A was filed, and the wash fastness problems related to the use of the pyrazole primaries still were unknown. Notably, U.S. Pat. No. 5,364,415A does not deal at all with wash fastness.

Once the vat dye pigments are precipitated in the cortex of the hair after the re-oxidation is completed, an exceptional long-lasting hair coloration is achieved, caused by the absolute insolubility of the formed pigments. Hence, this type of coloration provides advanced long-lasting colorations which show superior wash fastness versus traditional direct dyes added to conventional oxidative hair colorants using primaries and couplers. After 15 or more washes with shampoo, the so-treated hair remains completely unchanged while the entire color load (oxidative coupling products and the vat dye(s), respectively) is still there with no traces of any loss. This observation is remarkable as the addition of solubilized vat dyes do not only add more brilliance to the conventional oxidative coloring or opens completely new color spaces which cannot be directly accessed only by using conventional oxidative hair colorants, but causes a significantly improved wash fastness. This is increasingly surprising as it is known that especially the vibrant red shades containing literature known pyrazole derivatives (e.g. Hydroxyethyl-4,5-diamino-pyrazol sulfate or Hexyl-4,5-diamino-pyrazol) show deficiencies in wash fastness and quick fading versus other shades is a known issue for the industry. Without wanting to be bound by theory, a plausible explanation obviously may be that the polycyclic aromatic structure of the indanthrene related vat dyes works like a closing door while being precipitated into the hair cortex. The vat dye can find a solid space in between the protein-based keratin strings and forms a composite type arrangement with the keratin. This disables other water soluble, hence flexible and moving molecules such as the coupling products formed by the reaction of primaries and couplers in conventional oxidative hair coloring, to move out of the hair, hence, the color will show improved resistance against shampooing. This proposed mechanism appears to be supported by known literature observations: DE10259033 (Langhals, Behrens, Bauer, Speckbacher) describes the arrangement of water soluble perylene dyes, which have very comparable polycyclic aromatic ring structures like the solubilized vat dyes, between multiple inorganic layer structures via intercalation of the chromophores while forming a herring bone pattern in a dedicated 45° angle in relation to the layers. Such arrangement has been demonstrated via X-ray analysis. This proposed concept appears to be further supported by the observation that the wash fastness properties are improved if the conventional oxidative dyes are applied prior to the vat dyes or simultaneously. If the solubilized vat dyes are applied first followed by treatment with conventional oxidative hair dyes, the color result caused by the addition of the conventional oxidative hair color is only very moderate and the wash fastness of these is very poor and significantly less compared to an application without any treatment of solubilized vat dyes. This observation explains that is the vat dyes are applied first, the available positions are already effectively blocked with the precipitated vat dyes, thereby not leaving sufficient space for conventional oxidative dyes to penetrate into the hair cortex. The positive effect of an improved wash fastness is observed as expected when proceeding vice versa, hence applying the conventional oxidative dyes prior to or simultaneously with the application of the solubilized vat dyes.

Another observation made by the present inventors is that the structure and size of the (solubilized) polycyclic aromatic structure of the vat dye has an impact of the wash fastness obtained, or the improvement thereof. As regards the structure, best results were obtained for vat dyes having a flatbed polycyclic aromatic structure, as opposed to structures having a “kink”, such as in vat blue 4 (CAS #2747-19-5) or vat blue 6 (CAS #2519-28-0).

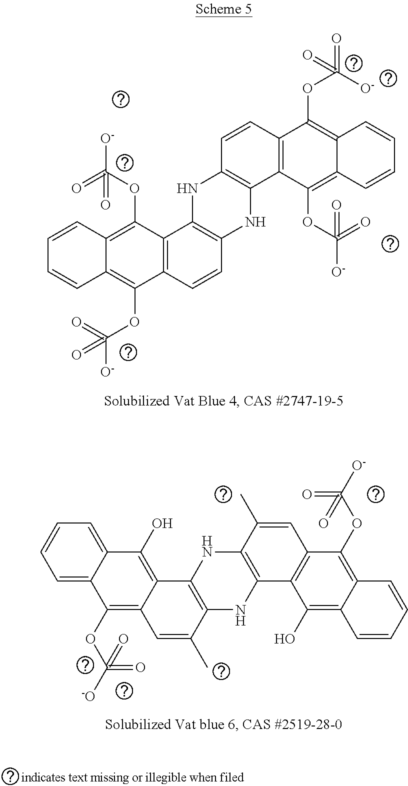

As regards the size of the flatbed polycyclic aromatic structure, the present inventors found out that a series of aromatic rings such as in anthracene or higher derivatives thereof such as tetracene or pentacene does not give the optimal results. Rather, it is understood that for achieving optimal results a polycyclic aromatic flatbed chromophore structure of four or more polycondensed rings is required, which flatbed chromophore structure optionally may comprise one or more nitrogen atoms, and includes a pyrene structural element according to Formula (4):

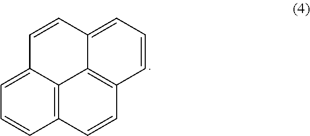 [SMILES: c1cc2ccc3cccc4ccc(c1)c2c34]

As an upper limit for achieving optimal results, it is understood that a polycyclic aromatic flatbed chromophore structure of at most eight polycondensed rings is required, optionally comprising one or more nitrogen atoms, which flatbed chromophore structure optionally may comprise one or more nitrogen atoms, and includes a pyranthrene structural element according to Formula (5):

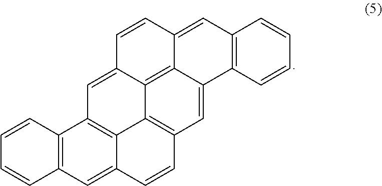 [SMILES: c1ccc2c(c1)cc1ccc3cc4c5ccccc5cc5ccc6cc2c1c3c6c54]

Optimal results are believed to be achieved with a polycyclic aromatic flatbed chromophore structure of six polycondensed rings optionally comprising one or more nitrogen atoms, said flatbed chromophore structure including a dibenzo-pyrene structural element according to Formula (6):

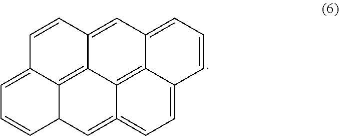

According to particular embodiments, composition B comprises a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof. Vat yellow 1 and vat orange 9 exhibit a polycyclic aromatic flatbed chromophore structure of eight polycondensed rings according to Formula (5). Vat yellow 4 and vat orange 3 exhibit a polycyclic aromatic flatbed chromophore structure of six polycondensed rings according to Formula (6), which is presently considered the optimal size of the flatbed structure for improving the wash fastness.

The improved wash fastness achieved according to the teachings of the present invention is evaluated by determining the resulting hair color “as obtained”, i.e. prior to any washing, determining the resulting hair color after 15 standard wash cycles, and determining the color difference. By comparing the color difference obtained after coloring the hair with the oxidative dye system alone prior to washing//after 15 wash cycles with the color difference obtained after coloring the hair with the oxidative dye system plus vat dyes prior to washing//after 15 wash cycles, a factor may be obtained which is an indication of the wash fastness improvement obtained.

It is noted that the factor obtained is an approximation indicating a relative improvement of the wash fastness, since the evaluation involves comparing two different color shades (with and without the color imparted by the vat dyes). The evaluation accordingly cannot give accurate results, and the factor obtained is a qualitative indication but not a quantitative indication. In order to exclude any impact of the original hair color (prior to any coloring), the evaluation is carried out using white natural hair.

The wash fastness improvement in other words may be described as a ratio ΔE(OXID)/ΔE(OXID+VAT), wherein:
ΔE(OXID) is the color difference [(L2−L1)²+(a2−a1)²+(b2−b1)²]^{1/2},
ΔE(OXID+VAT) is the color difference [(L4−L3)²+(a4−a3)²+(b4−b3)²]^{1/2},
L1, a1 and b1 are the CIELAB L*a*b color coordinates, determined on natural white hair, colored with composition A alone, prior to washing,
L2, a2 and b2 are the CIELAB L*a*b color coordinates, determined on natural white hair, colored with composition A alone, after 15 standard wash cycles,
L3, a3 and b3 are the CIELAB L*a*b color coordinates, determined on natural white hair, colored with compositions A and B, prior to washing,
L4, a4 and b4 are the CIELAB L*a*b color coordinates, determined on natural white hair, colored with compositions A and B, after 15 standard wash cycles.

According to the present invention, the ratio ΔE(OXID)/ΔE(OXID+VAT) preferably is 1.5 or more. According to embodiments, the ratio is 2.0 or more, or even 5.0 or more.

A standard wash cycle is defined as rinsing the colored hair tress with luke warm water (25° C.) for 10 seconds. A portion of ca. 10 g commercial shampoo (e.g. SP color save shampoo) is applied to the wet tress and gently rubbed in for 30 seconds, followed by rinsing with luke warm water for another 30 seconds. The tress in then gently dried with a towel followed by drying with a blow dryer for 2 minutes. Subsequently, the procedure is then repeated 14 times to achieve a total of 15 wash cycles.

The colors are measured with a suitable spectrophotometer, for example with a Konica Minolta portable spectrophotometer CM-26D, in order to determine the CIELAB L*a*b color coordinates.

The color produced according to the present invention may be in the color space range selected from orange, reddish-orange, copper, mahogany red, mahogany brownish, or vibrant red.

### 4 Method for Coloring a Keratinous Substrate

Subject matter of the present invention is a method for coloring a keratinous substrate. According to embodiments, the keratinous substrate is mammalian hair, in particular human hair such as human scalp hair. The method comprises:
a1 applying a composition A to said keratinous substrate, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof,
a2 applying a composition B to said keratinous substrate, said composition B comprising an aqueous medium and a solubilized vat dye dissolved in said aqueous medium,
b subsequent to step a2, applying a composition C to said keratinous substrate, said composition C comprising an oxidizing agent.

As described above, composition A comprises an oxidative dye system comprising oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, while composition B comprises one or more solubilized vat dyes.

The preferred order of application is first applying composition A to said keratinous substrate, followed by applying composition B, or applying compositions A and B simultaneously to the keratinous substrate. If desired, compositions A and B may be mixed prior to applying to the keratinous substrate, and the mixture be applied to the keratinous substrate. The reverse order—i.e. first applying composition B to said keratinous substrate, followed by applying composition A—is of course technical feasible, but not preferred. As already noted above, the reverse order of application entails only to a lesser extent the intended improvement of the wash fastness.

Composition A preferably exhibits a pH in the mild alkaline range, i.e. a pH in the range of 8.0-11.0, in particular in the range of 8.5-9.5, such as a pH of about 9.0, which is the usual pH range used for oxidative dyeing with oxidative colorants using primaries and couplers. Similarly, also composition B preferably exhibits a pH in the range of 8.0-11.0, in particular in the range of 8.5-9.5, such as a pH of about 9.0. This allows maintaining the ph within a range particularly suitable for swelling the keratinous substrate and established for oxidative dye uptake.

After applying compositions A and B, the oxidative dye precursors and solubilized vat dyes are allowed to penetrate into the cortex of the keratinous substrate, in particular human hair, preferably under mild alkaline conditions. This phase also may be referred to herein as a “penetration phase”. In order to do so, compositions A and B are maintained on the keratinous substrate for a sufficient duration, preferably for at least 20 min, in particular 20-40 min. Longer penetration phases of course are technically feasible, but are subject to consumer compliance in a hair dresser salon. Shorter penetration phases may result in incomplete penetration of the oxidative dye precursors and/or solubilized vat dyes into the cortex of the keratinous substrate. The penetration phase typically involves a temperature of 20-40° C., in particular 35-40° C. The pH during the penetration typically is in the range of 8.0-11.0, in particular in the range of 8.5-9.5, such as a pH of about 9.0.

If desired, the penetration of the oxidative dye precursors and solubilized vat dyes into the cortex of the keratinous substrate (such as human hair) may be enhanced by incorporating a penetration enhancer into composition A, composition B, or both. Obviously, a penetration enhancer could as well be incorporated separately, without being a component of composition A or B. According to embodiments, the penetration enhancer is selected from ammonia, urea, monoethanolamine, 2-amino-1-propanol, or a combination thereof.

During the penetration phase, it is important to keep the sulfonic ester structure intact to avoid or at least keep within acceptable limits any early oxidation of the solubilized vat dye as this may distract the penetration process, and may lead to incomplete use of the applied dye amount (which in turn may result in an unsatisfactory color result). In order to prevent insetting oxidation of the solubilized vat dyes at that point in time, a reducing agent may be added to the coloring mixture (in particular to composition B). Examples of suitable reducing agents comprise sodium sulfite, citric acid, ascorbic acid and similar mild reducing agents, used alone or in combination.

The penetration of the oxidative dye precursors and solubilized vat dyes into the cortex of the keratinous substrate may be further enhanced by applying a heat treatment (during or) following the penetration phase. In particular, such heat treatment may comprise treating the keratinous substrate with a flat iron having temperature of 80-230° C. Typical heat treatment durations are up to 30 seconds, in particular 2-20 seconds. Longer durations of the heat treatment (if any) are possible, but care should be taken in order to avoid (or at least limit) any potential damage to the keratinous substrate such as human hair.

As noted previously, the oxidative dye precursors and solubilized vat dyes are allowed during the penetration phase to penetrate into the cortex of the keratinous substrate, in particular human hair. It has been observed that lowering the alkaline pH down to for example pH=6 is not beneficial as it will hinder the solubilized vat dye to penetrate and doing so will decrease the swelling of the keratin fibers, which swelling needs mild alkaline conditions. Further, there is no need to force the sulfonic ester to cleave by lowering the pH to gentle acidic conditions. Rather, it has been observed that the bulky aromatic chromophore systems deeply flow into the cortex of the swollen hair also without an acidifying step as the cleavage of the sulfonic ester group appears to a large extent due to the lability of the sulfonic ester in aqueous media per se.

Following the penetration phase and the optional heat treatment, the oxidative dye precursors and leuco vat dyes are oxidized to the respective oxidative dyes and vat dyes, by applying composition C to the keratinous substrate, which composition C comprises an oxidizing agent. Any oxidizing procedure can be applied. This may include very gentle conditions using for example hydrogen peroxide solutions from 1.0-12.0 vol %. Common in the hair color industry are the following typical concentrations: 1.9, 6.0, 9.0 or 12.0 vol %. Composition C typically has a pH in the range of 2.0-4.0, in particular in the range of 2.5-3.5. The pH of composition C does not result in acidifying, however. When mixed with compositions A and B, the pH overall is maintained in the range from 7.0-10.0, typically in the range from 8.0-10.0, for example in the range of from 8.5-9.5. Composition C is maintained on the keratinous substrate preferably for less than 30 min, in particular 10-20 min, at a temperature of 20-40° C., in particular 35-40° C., and pH=8.0-11.0.

It is further preferred that the coloring method is free of any acidifying step, so that during the application phase, penetration phase, optional heat treatment and the oxidizing treatment, the pH is maintained preferably in the range of from 7.0-10.0, typically in the range from 8.0-10.0, for example in the range of from 8.5-9.5.

The method may further comprise any usual post-treatment steps. For example, the keratinous substrate is usually rinsed after completing the oxidizing phase, which rinsing is typically done with luke warm water, or with water having a temperature of about 37° C. to slightly above 37° C. Optional further post-treatment(s) may comprise for example shampooing, conditioning, drying the keratinous substrate, or a combination thereof.

Any of compositions A, B, C further may comprise usual additives such as pH adjusters, chelating agents, fragrances, care components, texture ingredients, medium reagents, solvents, surfactants, or a combination thereof.

### 4.1 the Oxidative Dye Precursors in Composition A

Composition A comprises an oxidative dye system as described above. All embodiments, examples and additional information described above in chapter 1 as well apply to composition A as used in the method of the present invention. For the sake of completeness, the following information is repeated.

Composition A comprises a pyrazole primary and a coupler, wherein the pyrazole primary comprises a 4,5-diaminopyrazole, an aminopyrazolopyridine, a ring-condensed 4,5-diaminopyrazolone, or a combination thereof.

According to preferred embodiments, composition A comprises a 4,5-diaminopyrazole according to Formula (1):

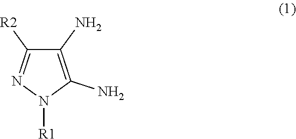

wherein R1 is linear or branched C1-C6 (hydroxy)alkyl and R2 is H or linear or branched C1-C6 (hydroxy)alkyl.

According to particular embodiments, the pyrazole primary in composition A comprises a 4,5-diaminopyrazole selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.

According to embodiments, composition A comprises an aminopyrazolopyridine according to Formula (2):

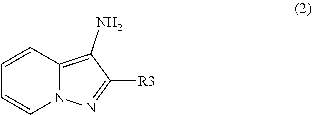

wherein R3 is (2-hydroxyethyl)oxy or 4,4-dimethylpiperazinium, or a combination thereof.

According to embodiments, composition A comprises a ring-condensed 4,5-diaminopyrazolone according to Formula (3):

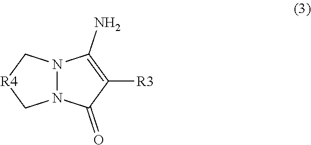

wherein R4 is methylene, ethylene, propylene, or a combination thereof.

According to preferred embodiments, composition A comprises a pyrazole primary selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, 2,3-diaminodihydropyrazolopyrazolone (CAS #857035-94-0), dimethylpiperaziniumaminopyrazolopyridine (CAS #1256553-33-9), 3-Amino-2-((2-hydroxyethyl)oxy)-Pyrazolo[1,5−a]pyridin (CAS #1079221-49-0), or a combination thereof. According to yet more particular embodiments, composition A comprises a pyrazole primary selected from 4,5-diamino-1-methylpyrazol, 4,5-diamino-1-ethylpyrazol, 4,5-diamino-1-hexylpyrazol, 4,5-diamino-1-hydroxyethylpyrazol, or a combination thereof.

Typically, composition A will comprise—in addition to the pyrazole primaries-one or more conventional primaries. According to particular embodiments, the conventional primary intermediates optionally present in composition A may comprise N,N-bis(2-hydroxyethyl)-p-phenylenediamine, 4-aminophenol, p-phenylenediamine, 3-hydroxypropyl-p-phenylenediamine, 2-methyl-p-phenylenediamine, 2-methoxymethyl-p-phenylenediamine, or a combination thereof.

According to particular embodiments, composition A comprises one or more couplers selected from resorcinol, 2-methylresorcinol, hydroxyethyl-3,4-methylenedioxyaniline, m-aminophenol, 1-naphthol, 2-amino-4-hydroxyethylaminoanisole, 4-amino-2-hydroxytoluene, 2-methyl-5-hydroxyethylaminophenol, phenyl methyl pyrazolone, 6-hydroxy-benzomorpholine, 6-methoxy-2-methylamino-3-aminopyridine, or a combination thereof.

According to embodiments, composition A comprises each primary in a concentration of 0.005-6.0 wt %, in particular 0.01-3.0 wt %, such as 0.2-2.0 wt %, based on the weight of composition A. According to embodiments, composition A comprises each coupler in a concentration of 0.005-6.0 wt %, in particular 0.01-3.0 wt %, such as 0.2-2.0 wt %, based on the weight of composition A.

Typically, composition A comprises a total amount of oxidative dye precursors, namely the one or more couplers, the one or more pyrazole primaries, and the one or more optional conventional primary intermediates, up to 12.0 wt %, or from 0.1-12.0 wt %, or from 0.2-10.0 wt %, or from 0.5-8.0 wt % by total weight of composition A.

### 4.2 the Solubilized Vat Dyes in Composition B

Composition B comprises one or more solubilized vat dyes as described above. All embodiments, examples and additional information described above in chapters 2 and 3 as well apply to composition B as used in the method of the present invention. For the sake of completeness, the following information is repeated.

According to the present invention, composition B comprises an aqueous medium and a solubilized vat dye dissolved in said aqueous medium. According to embodiments, composition B comprises two or more solubilized vat dyes, for example three or four solubilized vat dyes. Typically, the solubilized vat dye in composition B is a leuco vat dye sulfonic ester, or a combination of two or more leuco vat dye sulfonic esters.

According to embodiments, composition B comprises a solubilized vat dye selected from solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CAS #12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), or a combination thereof.

According to particular embodiments, composition B comprises a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.

According to embodiments, composition B comprises each solubilized vat dye in a concentration of 0.50-2.0 wt %, in particular a concentration of 0.6-1.5 wt %, for example a concentration of 0.75-1.0 wt %, based on the weight of composition B. Typically, composition B comprises a total amount of solubilized vat dyes up to 8.0 wt %, or from 0.5-6.0 wt %, or from 1.0-5.0 wt %, or from 1.5-4.0 wt % by total weight of composition B.

According to preferred embodiments, at least one of the solubilized vat dyes in composition B exhibits a polycyclic aromatic flatbed chromophore structure according to Formula (4), (5) and/or (6), as described above in chapter 3.

The wash fastness improvement achieved by the solubilized vat dyes in composition B preferably meets the ratio ΔE(OXID)/ΔE(OXID+VAT) described in chapter 3, wherein the ratio is 1.5 or more.

### 4.3 Composition A-Formulation Details

Composition A is suitable for delivering the oxidative dye precursors to the hair, is compatible with composition B, and preferably has a rheology and viscosity such that it can be utilised by hair salon professionals. Suitable formulations comprise so-called gel-network systems such as described for example in EP2308564A1), and oil-based compositions such as described for example in EP2338572A1. According to embodiments, composition A preferably is free of inorganic pigments, parabens, silicones, or a combination thereof.

### 4.3.1 Gel-Network Systems

Gel-network thickener systems are defined as a thickening system comprising a tertiary component system. This system comprises first anionic surfactant component selected from C14 to C30 alkyl phosphate, C14 to C30 alkyl ether phosphate and or mixtures thereof, a second component selected from C14 to C30 fatty alcohols and a third non-ionic surfactant component selected from polyoxyethylene C14 to C30 alkyl ethers. Those skilled in the art will recognize that gel network thickener systems usually have a complex structure of networked lamellar bi-layers and/or vesicles and sometimes crystals. These systems usually have creamy appearance and feel and are thus particularly desirable.

The first anionic surfactant component of the gel network thickener system is selected from C8 to C30, preferably C14 to 18 alkyl phosphate, C8 to C30 preferably C14 to C18 alkyl ether phosphate or mixtures thereof. Preferably the alkyl ether phosphates have an average 1 to 20, preferably 1 to 10 and most preferably 1 to 3 ethylene oxide units. According to embodiments, composition A comprises at least 1.0 wt % of said first surfactant component, preferably from 1.0-3.0 wt %, more preferably from 1.2-2.0 wt % of said first surfactant component, based on the weight of composition A. The first surfactant component assists in the formation of the gel network and is preferably utilized at the above levels to assist the maintenance of the desired rheology range particularly at the upper value to prevent excessive stickiness.

The gel network system further comprises as a second component, a linear or branched C14 to C30 fatty alcohols. Most preferably the second component is selected from cetyl, stearyl, cetostearyl or behenyl alcohols or mixtures thereof. Composition A may comprise from 2.0-8.0 wt %, preferably from 4.0-6.0 wt % of said fatty alcohol based on the weight of composition A. The second component assists in the stabilization of the gel network system and also assists in the maintenance of the desired rheology range particularly at the upper value to prevent excessive stickiness.

The third surfactant component of the gel network thickener system is a non-ionic surfactant, selected from polyoxyethylene C14 to C30 alkyl ethers, comprising one or more polyethyleneoxide chains, preferably having at least about 25, preferably from about 50 to 200, most preferably from about 100 to 200 ethylene oxide units. Suitable surfactants include ceteareth-25, steareth-100, steareth-150, steareth-200 and mixtures thereof. The third surfactant component acts as a co emulsifier and stabilizer of the gel network system. Moreover, the third surfactant assists in the formation of a soft and smooth composition. The third surfactant component of the gel network thickener system may be present in an amount from 0.1-5.0 wt % of composition A.

More than one surfactant and or component of each of the above specified types of the surfactants or components can be used in the gel network. Composition A, if formulated as a gel-network system, may comprise a total amount of said first surfactant component, said second surfactant component and said third surfactant component of at least 3.0 wt %, preferably at least 4.0 wt %, more preferably at least 5.0 wt %, based on the weight of composition A.

### 4.3.2 Oil-Based Compositions

When formulated as an oil-based composition, composition A may contain a semi-solid mixture of hydrocarbons (with carbon numbers mainly higher than 25) and fatty alcohols. Hydrocarbons, for example liquid petroleum jelly preferably, is present in an amount from 10.0.-65.0 wt %, preferably 15.0-60.0 wt %. One or more C12-C34 fatty alcohols, for example lauryl alcohol, cetyl alcohol, stearyl alcohol, oleyl alcohol, preferably are present in an amount from 5.0-30.0 wt %, preferably 8.0-20.0 wt %. Fatty alcohols comprising a C16-C30 linear alkyl chain are preferred.

When formulated as an oil-based composition, composition A preferably further comprises one or more nonionic surfactants. Examples of suitable non-ionic surfactants comprise POE/POP/POE (Poloxamer 184) or oxyethylenated C8-C30 alcohols such as oleyl alcohol (100E) and mixtures thereof, in a range of 5.0-15.0 wt %, preferably 7.0-10.0 wt %.

Viscosity modifiers, in particular a viscosity modifier in a form of an organic derivative of hectorite clay, such as for example distearyldimethylammonium-modified hectorite, may be present in an amount from 0.5-6.0 wt %, preferably 1.0-3.0 wt %.

Solvents such as propylene glycol, ethanol, hexylene glycol, dipropylene glycol, or combinations thereof typically may be present in an amount from 2.0-15.0 wt %, preferably 3.0-8.0%. Antioxidant such as ascorbic acid typically may be present in an amount from 0.1-0.7 wt %.

Optional chelants, which may be identical or different, typically are selected from the group consisting of diethylenetriamine-N,N′,N″-polyacids, diethylenetriaminepentaacetic acid (DTPA), diethylenetriaminepenta(methylene phosphonic acid) (DTPMP), ethylene diamine tri(methylene phosphonate), hexamethylene diamine tetra(methylene phosphonate), ethylenediaminedisuccinic acid (EDDS), their salts thereof, their derivatives thereof, and mixtures thereof. A total amount of chelants typically ranges from at least 0.01 wt %, preferably from 0.01-5 wt %, more preferably from 0.25-3 wt %, even more preferably from 0.5-2 wt %.

Alkalizing agents as monoethanolamine or ammonium, sodium hydroxide, typically may be present in a range from 2.0-12.0 wt %. Suitable pH modifiers and/or buffering agents may include, but are not limited to ammonia, acidulents (such as inorganic and inorganic acids including for example phosphoric acid, acetic acid, ascorbic acid, citric acid or tartaric acid, hydrochloric acid); alkanolamines (such as monoethanolamine, diethanolamine, triethanolamine, monopropanolamine, dipropanolamine, tripropanolamine, 2-amino-2-methyl-1-propanol, 2-amino-1-propanol, and 2-amino-2-hydroxymethyl-1,3,-propanediol); guanidium salts, alkali metal such as sodium hydroxide, tetrasodium pyrophosphate or ammonium hydroxides and carbonates; and mixtures thereof.

### 4.4 Composition B-Formulation Details

Composition B is suitable for delivering the solubilized vat dyes to the hair, is compatible with composition A, and preferably has a rheology and viscosity such that it can be utilised by hair salon professionals. Suitable formulations comprise gel-like systems, and shampoo-like systems. According to embodiments, composition B preferably is free of inorganic pigments, parabens, silicones, or a combination thereof.

### 4.4.1 Gel-Like Compositions

When formulated as a gel-like composition, composition B comprises 10.0-30.0 wt % of one or more saturated or unsaturated fatty acids. According to embodiments, composition B comprises 12.0-25.0 wt %, in particular 15.0-20.0 wt % of said one or more saturated or unsaturated fatty acids. Suitable fatty acids for formulating composition B as a gel-like composition preferably exhibit a linear carbon chain length of C12 and more, in particular linear carbon chain length of C16-C30. Examples of fatty acids comprise palmitic acid (C16:0), stearic acid (C18:0), palmitoleic acid (C16:1), oleic acid (C18:1), linoleic acid (C18:2), linolenic acid (C18:3), or a combination thereof.

The gel-like composition preferably further comprises 2.0-20.0 wt % of a rheology agent, in particular wherein the rheology agent is selected from ammonia, monoethanolamine, or a combination thereof. According to preferred embodiments, the gel-like composition exhibits a creamy consistency.

### 4.4.2 Shampoo-Like Compositions

When formulated as a shampoo-like composition, composition B comprises 2.0-40.0 wt % anionic surfactant, 2.0-20.0 wt % amphoteric surfactant, and 0-10.0 wt % of an optional humectant.

According to embodiments, composition B when formulated as a shampoo-like composition comprises 4.0-25.0 wt %, in particular 6.0-15.0 wt % anionic surfactant, 3.0-15.0 wt %, in particular 4.0-10.0 wt % amphoteric surfactant, and 1.0-8.0 wt % of humectant.

According to embodiments, the anionic surfactant is selected from alkyl sulfates, alkyl ether sulfates, alkylamido ether sulphates, monoglyceride sulphates, a-olefin sulphonates, paraffin sulphonates, alkyl phosphates, alkyl sulphoacetates, acyl isethionates, alkyl ether phosphates, alkyl phenyl ether sulfonates, N-cocoyl fatty acyl glutamate, N-lauroyl glutamate, N-acylamino acid salts, N-coconut oil fatty acyl glycine, N-coconut oil fatty acyl alanine salts, salts of alkyl sulfosuccinic acids, and a combination thereof.

According to embodiments, the amphoteric surfactant is selected from betaines, in particular alkyl amido betaines, alkylbetaines, amidosulfobetaine, carboxybetaine-type betaines, sulfobetaine-type betaines, hydroxysulfobetaine betaines, phospobetaine-type betaines, and a combination thereof.

According to embodiments, the optional humectant is selected from glycerol, glycerol esters, propylene glycol, isopentyldiol, and a combination thereof.

As a particular example for a composition B when formulated as a shampoo-like composition, composition 8 may comprise 6.0-10.0 wt % Sodium Laureth Sulfate, 4.0-6.0 wt % Cocoamidopropyl Betaine, and 2.0-4.0 wt % glycerin.

### 4.5 Composition C-Formulation Details

Composition C comprises an oxidizing agent. In particular, composition C may comprise hydrogen peroxide, typically in a concentration from 1.0-12.0 vol %. Composition C preferably has a pH in the range of 2.0-4.0, in particular in the range of 2.5-3.5. Composition C further may comprise pH adjusters, chelating agents, fragrances, care components, texture ingredients, medium reagents, solvents, surfactants as needed. According to embodiments, composition C preferably is free of inorganic pigments, parabens, silicones, or a combination thereof. Composition C may be, for example, a commercially available hydrogen peroxide solution such as the composition marketed by Wella under the brand name WELLOXON Perfect.

### 5 Ready-to-Use Hair Coloring Formulation

Further subject matter of the present invention is a ready-to-use hair coloring formulation. The ready-to-use hair coloring formulation is obtained by mixing composition A as described above with composition B described above. Composition A comprises an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof. Composition B comprises an aqueous medium and a solubilized vat dye dissolved in said aqueous medium.

The ready-to-use hair coloring formulation preferably exhibits a pH in the mild alkaline range, i.e. a pH in the range of 8.0-11.0, in particular in the range of 8.5-9.5, such as a pH of about 9.0.

The ready-to-use hair coloring formulation may comprise a penetration enhancer, in particular a penetration enhancer selected from ammonia, urea, monoethanolamine, 2-amino-1-propanol, or a combination thereof.

The oxidative dye precursors in the ready-to-use hair coloring formulation, and in particular the pyrazole primary, are derived from composition A. The oxidative dye system accordingly is as described above in particular in chapter 1. The solubilized vat dyes in the ready-to-use hair coloring formulation are derived from composition B. The solubilized vat dye system accordingly is as described above in particular in chapters 2 and 3.

With respect to the amounts of oxidative dye precursors and solubilized vat dyes, the ready-to-use hair coloring formulation preferably comprises each primary in a concentration of 0.0025-3.0 wt %, each coupler in a concentration of 0.0025-3.0 wt %, and each solubilized vat dye in a concentration of 0.25-1.0 wt %, with all wt %-ranges being based on the total weight of the formulation. According to embodiments, the ready-to-use hair coloring formulation may comprise each primary in a concentration 0.005-1.5 wt %, each coupler in a concentration of 0.005-1.5 wt %, and each solubilized vat dye in a concentration of 0.3-0.75 wt %, with all wt %-ranges being based on the total weight of the formulation. The ready-to-use hair coloring formulation may comprise a total amount of oxidative dye precursors (couplers, pyrazole primaries and conventional primaries) up to 6.0 wt %, and a total amount of solubilized vat dyes up to 4.0 wt %, with all wt %-ranges being based on the total weight of the formulation.

The above weight amount ranges of oxidative dye precursors and solubilized vat dyes imply that for obtaining the ready-to-use hair coloring formulation, composition A and composition B are mixed in similar or equal amounts, i.e. in a weight ratio of about 1:1. The ratio of compositions a and B is not critical, however, and it is possible to obtain the ready-to-use hair coloring formulation mixing compositions a and B in deviating weight ratios, for example within a range A: 8 from 1:5 to 5:1. It is understood, however, that when using compositions A and B in weight ratios other than 1:1, the amounts of oxidative dye precursors and solubilized vat dyes in compositions A and B preferably are adapted to meet the above indicated weight limitations.

### 6 Kit for Coloring Hair

Further subject matter of the present invention is a kit for coloring hair. The kit comprises in in one compartment (or container) a composition A as described in chapter 1 above, and in a separate compartment (or container) a composition B as described in chapters 2-3 above. Composition A comprises an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof. Compartment B comprises a composition B, said composition B comprising an aqueous medium and a solubilized vat dye dissolved in said aqueous medium.

According to embodiments, the kit further may comprise another separate compartment (or container), which further separate compartment (or container) comprises a composition C as described above. Composition C comprises an oxidizing agent, in particular hydrogen peroxide, typically in a concentration from 1.0-12.0 vol %. Composition C may be, for example, a commercially available hydrogen peroxide solution such as the composition marketed by Wella under the brand name WELLOXON Perfect.

### 7 Hair Coloring Component

Further subject matter of the present invention is a hair coloring component. The hair coloring component is intended for use together with a commercial hair color and oxidizing solution, for example commercially available creme hair color marketed by Wella under the brand name KOLESTON Perfect and commercially available Hydrogen peroxide solution marketed by Wella under the brand name WELLOXON Perfect.

### 7.1 Gel-Like Hair Coloring Component

A gel-like hair coloring component according to the present invention comprises one or more solubilized vat dyes, each in a concentration of 0.50-2.0 wt %, 10.0-30.0 wt % of one or more saturated or unsaturated fatty acids, and 50-89.50 wt % water, wherein wt % each is based on the weight of the component.

The solubilized vat dyes are as described above in chapters 2-3. The gel-like hair coloring component comprises each solubilized vat dye in a concentration of 0.50-2.0 wt %, in particular a concentration of 0.6-1.5 wt %, for example a concentration of 0.75-1.0 wt %, based on the weight of the gel-like hair coloring component. Typically, the gel-like hair coloring component comprises a total amount of solubilized vat dyes up to 8.0 wt %, or from 0.5-6.0 wt %, or from 1.0-5.0 wt %, or from 1.5-4.0 wt % by total weight of the gel-like hair coloring component.

According to embodiments, the gel-like hair coloring component comprises 12.0-25.0 wt %, in particular 15.0-20.0 wt % of said one or more saturated or unsaturated fatty acids. Suitable fatty acids preferably exhibit a linear carbon chain length of C12 and more, in particular linear carbon chain length of C16-C30. Examples of fatty acids comprise palmitic acid (C16:0), stearic acid (C18:0), palmitoleic acid (C16:1), oleic acid (C18:1), linoleic acid (C18:2), linolenic acid (C18:3), or a combination thereof.

According to embodiments, the gel-like hair coloring component preferably further comprises 2.0-20.0 wt % of a rheology agent selected from ammonia, monoethanolamine, or a combination thereof. According to preferred embodiments, the gel-like hair coloring component exhibits a creamy consistency.

The gel-like hair coloring component further may comprise a penetration enhancer, in particular a penetration enhancer selected from ammonia, urea, monoethanolamine, 2-amino-1-propanol, or a combination thereof (if not already present as a rheology agent).

The gel-like hair coloring component further may comprise a reducing agent, in particular sodium sulfite, citric acid, ascorbic acid or a combination thereof.

The gel-like hair coloring component further may comprise one or more components selected from pH adjusters, chelating agents, fragrances, care components, texture ingredients, medium reagents, solvents, nonionic surfactants.

### 7.2 Shampoo-Like Hair Coloring Component

A shampoo-like hair coloring component according to the present invention comprises one or more solubilized vat dyes, each in a concentration of 0.50-2.0 wt %, 2.0-40.0 wt % anionic surfactant, 2.0-20.0 wt % amphoteric surfactant, 0-10.0 wt % of an optional humectant, and 50.0-95.50 wt % water, wherein wt % each is based on the weight of the component.

The solubilized vat dyes are as described above in chapters 2-3. The shampoo-like hair coloring component comprises each solubilized vat dye in a concentration of 0.50-2.0 wt %, in particular a concentration of 0.6-1.5 wt %, for example a concentration of 0.75-1.0 wt %, based on the weight of the gel-like hair coloring component. Typically, the shampoo-like hair coloring component comprises a total amount of solubilized vat dyes up to 8.0 wt %, or from 0.5-6.0 wt %, or from 1.0-5.0 wt %, or from 1.5-4.0 wt % by total weight of the gel-like hair coloring component.

According to embodiments, the shampoo-like hair coloring component comprises 4.0-25.0 wt %, in particular 6.0-15.0 wt % anionic surfactant, 3.0-15.0 wt %, in particular 4.0-10.0 wt % amphoteric surfactant, and 1.0-8.0 wt % of humectant.

According to embodiments, the anionic surfactant is selected from alkyl sulfates, alkyl ether sulfates, alkylamido ether sulphates, monoglyceride sulphates, a-olefin sulphonates, paraffin sulphonates, alkyl phosphates, alkyl sulphoacetates, acyl isethionates, alkyl ether phosphates, alkyl phenyl ether sulfonates, N-cocoyl fatty acyl glutamate, N-lauroyl glutamate, N-acylamino acid salts, N-coconut oil fatty acyl glycine, N-coconut oil fatty acyl alanine salts, salts of alkyl sulfosuccinic acids, and a combination thereof.

According to embodiments, the amphoteric surfactant is selected from betaines, in particular alkyl amido betaines, alkylbetaines, amidosulfobetaine, carboxybetaine-type betaines, sulfobetaine-type betaines, hydroxysulfobetaine betaines, phospobetaine-type betaines, and a combination thereof.

According to embodiments, the optional humectant is selected from glycerol, glycerol esters, propylene glycol, isopentyldiol, and a combination thereof.

A particular example of a shampoo-like hair coloring component comprises 6.0-10.0 wt % Sodium Laureth Sulfate, 4.0-6.0 wt % Cocoamidopropyl Betaine, and 2.0-4.0 wt % glycerin.

The shampoo-like hair coloring component further may comprise a penetration enhancer, in particular a penetration enhancer selected from ammonia, urea, monoethanolamine, 2-amino-1-propanol, or a combination thereof.

The shampoo-like hair coloring component further may comprise a reducing agent, in particular sodium sulfite, citric acid, ascorbic acid or a combination thereof.

The shampoo-like hair coloring component further may comprise one or more components selected from pH adjusters, chelating agents, fragrances, care components, texture ingredients, medium reagents, solvents, nonionic surfactants.

8 Use of Vat Dyes and/or of the Hair Coloring Components for Improving the Wash Fastness of Hair Color Produced by Oxidative Dyes

Further subject matter of the present invention is the use of solubilized vat dyes or, respectively, of the hair coloring component described in chapter 7 above, for improving the wash fastness of hair color produced by oxidative dyes, essentially as described above. According to embodiments, one or more of the oxidative dyes are derived from oxidative dye precursors comprising a pyrazole primary and a coupler, as described above, in particular in chapter 1. The solubilized vat dyes are as described above, in particular in chapters 2-3.

The use contemplated by the present invention relates in particular to hair color in the orange, reddish-orange, copper, mahogany red, mahogany brownish, or vibrant red color space.

The wash fastness improvement may be described as a ratio ΔE(OXID)/ΔE(OXID+VAT), wherein:
ΔE(OXID) is the color difference [(L2−L1)²+(a2−a1)²+(b2−b1)²]^{1/2},
ΔE(OXID+VAT) is the color difference [(L4−L3)²+(a4−a3)²+(b4−b3)²]^{1/2},
L1, a1 and b1 are the CIELAB L*a*b color coordinates, determined on natural white hair, colored with composition A alone, prior to washing,
L2, a2 and b2 are the CIELAB L*a*b color coordinates, determined on natural white hair, colored with composition A alone, after 15 standard wash cycles,
L3, a3 and b3 are the CIELAB L*a*b color coordinates, determined on natural white hair, colored with compositions A and B, prior to washing,
L4, a4 and b4 are the CIELAB L*a*b color coordinates, determined on natural white hair, colored with compositions A and B, after 15 standard wash cycles.

According to the present invention, the ratio ΔE(OXID)/ΔE(OXID+VAT) preferably is 1.5 or more. According to embodiments, the ratio is 2.0 or more, or even 5.0 or more.

A standard wash cycle is defined as rinsing the colored hair tress with luke warm water (about 25° C.) for 10 seconds. A portion of ca. 10 g commercial shampoo (e.g. SP color save shampoo) is applied to the wet tress and gently rubbed in for 30 seconds, followed by rinsing with luke warm water for another 30 seconds. The tress in then gently dried with a towel followed by drying with a blow dryer for 2 minutes. Subsequently, the procedure is then repeated 14 times to achieve a total of 15 wash cycles.

The colors are measured with a suitable spectrophotometer, for example with a Konica Minolta portable spectrophotometer CM-26D, in order to determine the CIELAB L*a*b color coordinates.

### Examples

The oxidative dyeing process using solubilized vat dyes in combination with conventional oxidative hair dyes essentially comprises 2 steps.

A Penetration of the Solubilized Vat Dyes into the Hair Cortex

This happens under mild alkaline conditions at pH=9 in the presence of ammonia, mono ethanol amine, 2-amino-1-propanol, or other penetration enhances such as urea, benzyl alcohol etc. The oxidative primaries and couplers are being added optionally already at this stage which saves valuable time for the hair stylist looking on the overall application time. For sure the first penetration step can be done voluntarily also without the addition of the oxidative primaries and couplers, while these ones can be added once the penetration time is completed. However, we have noticed that there is no benefit in doing so, hence, the recommendation clearly is to apply the mixtures (solubilized vat dyes and the conventional oxidative hair color comprising of primaries and couplers) together as the obvious advantage of saving valuable application time is clearly beneficial for the presented invention. At this point in time it is important to keep the sulfonic ester structure intact to avoid any early oxidation of the solubilized vat dye as this will distract the penetration process and will lead to incomplete use of the applied dye amount which will result in an unsatisfactory color result. In order to prevent the solubilized vat dyes to start the oxidation at that point in time, sodium sulfite, citric acid, ascorbic acid etc. as reducing agent is added to the coloring mixture. In contrast to U.S. Pat. No. 5,364,415 (Lewis et. al.), the color results are significantly more intense and brilliant once the penetration time is kept at 20-30 min. at 40° C. at pH=9. Moreover, the addition of sodium meta bisulfite as described in U.S. Pat. No. 5,364,415 (Lewis et. al.), to lower the alkaline pH given by the addition of ammonia or urea, down to pH=6 is not beneficial as it will hinder the solubilized vat dye to penetrate and it will decrease the swelling of the keratin fiber which needs mild alkaline conditions. As mentioned above, there is no need to force the sulfonic ester to cleave while lowering the pH to gentle acidic conditions. It has been observed that the bulky aromatic chromophore systems deeply flow into the cortex of the swollen hair also without an acidifying step as the cleavage of the sulfonic ester group appears to a large extent due to the lability of the sulfonic ester in aqueous media per se. This observation is greatly beneficial as it dramatically simplifies the practical application in a hair dresser salon.

B Oxidation to Precipitate the Insoluble Pigments into the Cortex of the Hair

Once the penetration phase is over, any oxidizing procedure can be applied. This includes very gentle conditions using hydrogen peroxide solutions from 1-12 vol %. Common in the hair color industry are the following typical concentrations: 1.9, 6, 9 or 12 vol %. The oxidation process to form the vat dye pigments from the leuco form and the simultaneously happening lifting process (while oxidizers keep also oxidizing natural melanin in the hair) is typically completed within 20 min. Hence, the overall treatment will last no longer than 1 h which does correspond well to current oxidative hair coloring applications with no increase of any complexity of the treatment. A significant advantage is that any formulations can totally skip the use of organic solvents and alcohols such as ethanol or iso-propyl-alcohol. In addition, due to the fact that all solubilized vat dyes show an absolute comparable behavior regarding an homogeneous dye take-up and general fastness properties, all solubilized vat dyes can be easily blended with each other in combinations with various pre-formulated shades derived from conventional oxidative hair colorants comprising primaries and couplers, so that formulators and hair stylists as well as retail customers as users may have the artistic freedom to create all kind of natural and fashionable shades without any boundaries.

### General Procedure

5 g of one exemplary color color carrier mass in the form of gel and a dye mixture, comprises oleic acid and/or oleate, has pH value of 7 to 12,0. In addition the carrier mass contains antioxidants ascorbic acid or sodium sulfite, thickening agent xanthan gum or diutan gum, polysaccharides may also be used, for example, glucans, modified and unmodified starches, amylose, amylopectin, glycogen, dextrans, celluloses and its derivatives(methylcelluloses, hydroxyalkylcelluloses, ethyl hydroxyethylcelluloses, and carboxymethylcelluloses), the complexing agents ethylenediaminetetraacetic acid (EDTA) or/and ethylenediamine-N,N′-disuccinic acid (EDDS), alkalizing agent ammonium or alkanolamines such as monoethanolamine (MEA) or aminomethylpropanol (AMP), care ingredients (aminoacids, cationized cellulose, polyquaternium), perfume oils.

The carrier color mass in a form of foaming liquid, comprises of anionic surfactant and examples include alkyl sulfates, polyoxyethylene alkyl ether sulfates, alkyl phosphates, alkyl phenyl ether sulfonates, N-cocoyl fatty acyl glutamate, N-lauroyl glutamate, N-acylamino acid salts, N-coconut oil fatty acyl glycine, N-coconut oil fatty acyl alanine salts and salts of alkyl sulfosuccinic acids and amphoteric surfactants amidosulfobetaine, carbobetaine-type, sulfobetaine-type, hydroxysulfobetaine and phospobetaine-type. The foaming liquid contains humectants (glycerin, propylenglycol, the composition contains humectants (glycerin, propylene glycol), thickening agent, preservative system (propylparabene, methylparabene, sodium benzoate). Mixture of choice (not limiting to that example) according to the tables below (schemes 6-7) is blended homogeneously with 5 g of a commercial oxidative dye cream selecting a pre-formulated color shade free of choice (e.g. Koleston Perfect by Wella Professionals © by Wella Company).

Scheme 6: gel-like composition//hair coloring component

| Raw Material | Concentration W/W % in batch |
|---|---|
| Solubilized vat dye | 0.75 |
| EDTA | 0.1 |
| Sodium sulfite | 0.1 |
| Water | 69.83 |
| Propylene Glycol | 7.0 |
| Diutan gum | 0.22 |
| Oleic acid | 17.8 |
| Ammonia | 4.2 |

Scheme 7: shampoo-like composition//hair coloring component

| Raw Material | Concentration W/W % in batch |
|---|---|
| Solubilized vat dye | 0.75 |
| Sodium Laureth Sulfate | 8.00 |
| Cocoamidopropyl Betaine | 5.00 |
| Water | 82.33 |
| Glycerin | 3.00 |
| Diutan gum | 0.20 |
| Propylparabene | 0.17 |
| Methylparabene | 0.20 |
| Sodium Benzoate | 0.35 |

Hair tresses are then placed on a plate and treated with this mixture and kept for 20 min. at 35-40° C. This penetration step is done at pH=9, as the alkaline medium is best for swelling the hair and is established as a global standard for the oxidative dye uptake. Then, the hair tresses are placed into an oxidation bath at 35-40° C., keeping pH=9 to allow optimal lift level, comprising standard commercial products, so called color developers, containing hydrogen peroxide. Typical contents are 1.9, 6, 9 and 12 vol %. The developer can be chosen in absolute freedom depending on the grade of lift one may desire. After 20 min., the hair tresses are taken out rinsed with luke warm water to completely remove any traces of oxidants, powdered particles etc. and washed with shampoo, followed by an after-treatment with a commercial conditioner and then dried with a blow dryer. This after caring treatment ensures that the hair cortex will be closed again and the hair is converted back to the original state with a closed and healthy surface. The same process can be also applied on head as described above.

In principal, all common additional components, not detailed out in the above disclosed exemplary formulation, which are a typical ingredients of an oxidative hair coloring mixture in addition to the above mentioned components can be applied as well with no exception and negative impact on the coloring result. This includes the following general ingredient groups: coloring agents (solubilized vat dyes), penetration enhancers/alkalizers (e.g. ammonia, ethanol amine, 2-amino-1-propanol, benzyl alcohol, urea etc.), reducing agents/radical scavengers (e.g. sodium sulfite, citric acid, ascorbic acid etc.), chelating agents (e.g. EDTA, EDDS, editronic acid) etc.), fragrances, care components (e.g. hydrolyzed keratin, dimethicone, polyquaternium types etc.) texture ingredients (e.g. dinutan gum, carbomer, coco amides, hydroxyethyl cellulose etc.), medium reagents (e.g. oleic acid, cetearyl alcohol, lanolin alcohol, glycerin stearate, propylene glycol etc.) and surfactants (e.g. lauryl ether sulfate, lauryl sulfo acetate etc.).

The following table (scheme 8) describes the obtained colorations using an oxidative dye system alone, or in combination with one solubilized vat dye, both directly after coloring and after 15 standard wash cycles. The oxidative dye system is Koleston Perfect, a brand name by Wella company. The oxidation level is 6% hydrogen peroxide. This lift level is most common in commercial, oxidative permanent hair coloring. All experiments are carried out using “natural white hair” and at a pH of about 9.0.

Scheme 8:

| Koleston | solubilized | # wash | | | |
|---|---|---|---|---|---|
| Perfect* | vat dye | cycles | L** | a** | b** |
| none | none | none | 78.43 | 3.44 | 27.76 |
| KP 55/46 | none | none | 7.64 | 35.82 | 13.02 |
| KP 55/46 | none | 15x | 5.81 | 37.77 | 9.87 |
| KP 55/46 | yellow 4 | none | 11.88 | 39.32 | 20.32 |
| KP 55/46 | yellow 4 | 15x | 11.77 | 39.11 | 20.13 |
| KP 77/46 | none | none | 22.13 | 51.73 | 38.60 |
| KP 77/46 | none | 15x | 23.01 | 51.94 | 43.52 |
| KP 77/46 | yellow 4 | none | 27.77 | 55.55 | 47.61 |
| KP 77/46 | yellow 4 | 15x | 28.20 | 54.36 | 48.47 |
| KP 77/44 | none | none | 19.94 | 47.03 | 34.22 |
| KP 77/44 | none | 15x | 24.06 | 50.97 | 41.33 |
| KP 77/44 | yellow 4 | none | 26.36 | 51.36 | 45.28 |
| KP 77/44 | yellow 4 | 15x | 29.03 | 53.94 | 49.89 |
| KP 8/45 | none | none | 16.67 | 44.46 | 28.58 |
| KP 8/45 | none | 15x | 19.95 | 47.63 | 34.24 |
| KP 8/45 | yellow 4 | none | 32.79 | 51.85 | 56.36 |
| KP 8/45 | yellow 4 | 15x | 32.17 | 47.49 | 55.29 |
| KP 99/44 | none | none | 36.41 | 54.10 | 52.86 |
| KP 99/44 | none | 15x | 36.24 | 49.35 | 40.58 |
| KP 99/44 | yellow 4 | none | 29.23 | 47.26 | 49.58 |
| KP 99/44 | yellow 4 | 15x | 32.86 | 47.72 | 51.38 |
| KP 6/45 | none | none | 3.99 | 23.50 | 6.72 |
| KP 6/45 | none | 15x | 5.81 | 29.19 | 9.87 |
| KP 6/45 | yellow 4 | none | 21.84 | 36.12 | 37.45 |
| KP 6/45 | yellow 4 | 15x | 20.34 | 37.22 | 34.90 |
| KP 5/07 | none | none | 0.69 | 4.20 | 1.04 |
| KP 5/07 | none | 15x | 1.63 | 9.69 | 2.65 |
| KP 5/07 | yellow 4 | none | 5.39 | 21.36 | 9.14 |
| KP 5/07 | yellow 4 | 15x | 6.51 | 22.55 | 11.07 |
| KP 7/77 | none | none | 2.30 | 12.60 | 3.81 |
| KP 7/77 | none | 15x | 4.69 | 19.74 | 17.23 |
| KP 7/77 | yellow 4 | none | 8.18 | 21.33 | 13.94 |
| KP 7/77 | yellow 4 | 15x | 7.80 | 22.04 | 13.29 |

*Koleston Perfect is a brand name by Wella Company
**Color is measured via standard L*a*b values:
L = a measure of lightness and intensity. Range: 0 = black, 100 = white
a = a measure of red and green tones. Red = positive values, Green = negative values
b = a measure of blue and yellow tones. Yellow = positive values, Blue = negative values

The following table (scheme 9) describes the obtained colorations using an oxidative dye system alone, or in combination with two solubilized vat dyes, both directly after coloring and after 15 standard wash cycles. The oxidative dye system is Koleston Perfect, a brand name by Wella company. The solubilized vat dyes are a combination of “yellow 4” with “violet 1” (“Y4/V1”) in a weight ratio of 1:1, or a combination of “yellow 4” with “green 1” (“Y4/G1”) in a weight ratio of 2:1. The oxidation level is 6% hydrogen peroxide. This lift level is most common in commercial, oxidative permanent hair coloring. All experiments are carried out using “natural white hair” and at a pH of about 9.0.

Scheme 9:

| Koleston | solubilized | # wash | | | |
|---|---|---|---|---|---|
| Perfect* | vat dye | cycles | L** | a** | b** |
| KP 6/45 | none | none | 3.99 | 23.50 | 6.72 |
| KP 6/45 | none | 15x | 5.81 | 29.19 | 9.87 |
| KP 6/45 | Y4/V1 | none | 15.63 | 37.28 | 26.79 |
| KP 6/45 | Y4/V1 | 15x | 16.84 | 36.48 | 28.88 |
| KP 77/44 | none | none | 19.94 | 47.03 | 34.22 |
| KP 77/44 | none | 15x | 24.06 | 50.97 | 41.33 |
| KP 77/44 | Y4/V1 | none | 28.09 | 51.94 | 48.28 |
| KP 77/44 | Y4/V1 | 15x | 30.04 | 50.68 | 51.64 |
| KP 8/45 | none | none | 16.67 | 44.46 | 28.58 |
| KP 8/45 | none | 15x | 19.95 | 47.63 | 34.24 |
| KP 8/45 | Y4/V1 | none | 33.95 | 50.10 | 58.16 |
| KP 8/45 | Y4/V1 | 15x | 32.18 | 48.14 | 55.25 |
| KP 77/44 | none | none | 19.94 | 47.03 | 34.22 |
| KP 77/44 | none | 15x | 24.06 | 50.97 | 41.33 |
| KP 77/44 | Y4/G1 | none | 27.36 | 51.21 | 47.01 |
| KP 77/44 | Y4/G1 | 15x | 28.88 | 51.26 | 49.64 |
| KP 55/46 | none | none | 7.64 | 35.82 | 13.02 |
| KP 55/46 | none | 15x | 5.81 | 37.77 | 9.87 |
| KP 55/46 | Y4/G1 | none | 7.51 | 35.61 | 12.80 |
| KP 55/46 | Y4/G1 | 15x | 7.19 | 35.07 | 12.24 |

*Koleston Perfect is a brand name by Wella Company
**Color is measured via standard L*a*b values:
L = a measure of lightness and intensity. Range: 0 = black, 100 = white
a = a measure of red and green tones. Red = positive values, Green = negative values
b = a measure of blue and yellow tones. Yellow = positive values, Blue = negative values

The following table (scheme 10) describes the obtained coloration differences AL, As and Ab, comparing the L′ab values of schemes 8 and 9 above as obtained directly after coloring, with the L*a*b values obtained after 15 standard wash cycles.

Scheme 10:

| Koleston | solubilized | | | | | ΔE |
|---|---|---|---|---|---|---|
| Perfect | vat dye | ΔL* | Δa* | Δb* | ΔE** | ratio*** |
| KP 55/46 | none | −1.8300 | 1.9500 | −3.1500 | 4.1321 | 13.6008 |
| KP 55/46 | yellow 4 | −0.1100 | −0.2100 | −0.1900 | 0.3038 | |
| KP 77/46 | none | 0.8800 | 0.2100 | 4.9200 | 5.0025 | 3.2698 |
| KP 77/46 | yellow 4 | 0.4300 | −1.1900 | 0.8600 | 1.5299 | |
| KP 77/44 | none | 4.1200 | 3.9400 | 7.1100 | 9.1132 | 1.5396 |
| KP 77/44 | yellow 4 | 2.6700 | 2.5800 | 4.6100 | 5.9192 | |
| KP 8/45 | none | 3.2800 | 3.1700 | 5.6600 | 7.2693 | 1.6040 |
| KP 8/45 | yellow 4 | −0.6200 | −4.3600 | −1.0700 | 4.5320 | |
| KP 99/44 | none | −0.1700 | −4.7500 | −12.2800 | 13.1678 | 3.2291 |
| KP 99/44 | yellow 4 | 3.6300 | 0.4600 | 1.8000 | 4.0778 | |
| KP 6/45 | none | 1.8200 | 5.6900 | 3.1500 | 6.7536 | 2.1397 |
| KP 6/45 | yellow 4 | −1.5000 | 1.1000 | −2.5500 | 3.1563 | |
| KP 5/07 | none | 0.9400 | 5.4900 | 1.6100 | 5.7979 | 2.2927 |
| KP 5/07 | yellow 4 | 1.1200 | 1.1900 | 1.9300 | 2.5289 | |
| KP 7/77 | none | 2.3900 | 7.1400 | 13.4200 | 15.3879 | 14.8691 |
| KP 7/77 | yellow 4 | −0.3800 | 0.7100 | −0.6500 | 1.0349 | |
| KP 6/45 | none | 1.8200 | 5.6900 | 3.1500 | 6.7536 | 2.6547 |
| KP 6/45 | Y4/V1 | 1.2100 | −0.8000 | 2.0900 | 2.5441 | |
| KP 77/44 | none | 4.1200 | 3.9400 | 7.1100 | 9.1132 | 2.2314 |
| KP 77/44 | Y4/V1 | 1.9500 | −1.2600 | 3.3600 | 4.0841 | |
| KP 8/45 | none | 3.2800 | 3.1700 | 5.6600 | 7.2693 | 1.8498 |
| KP 8/45 | Y4/V1 | −1.7700 | −1.9600 | −2.9100 | 3.9297 | |
| KP 77/44 | none | 4.1200 | 3.9400 | 7.1100 | 9.1132 | 2.9997 |
| KP 77/44 | Y4/G1 | 1.5200 | 0.0500 | 2.6300 | 3.0381 | |
| KP 55/46 | none | −1.8300 | 1.9500 | −3.1500 | 4.1321 | 4.9122 |
| KP 55/46 | Y4/G1 | −0.3200 | −0.5400 | −0.5600 | 0.8412 | |

*difference in L*a*b values directly after coloring and after 15 wash cycles
**ΔE is the color difference [(ΔL)² + (Δa)² + (Δb)²]^{1/2}
***ΔE ratio is ΔE(OXID)/ΔE(OXID + VAT)

Scheme 10 above demonstrates that the improvement in wash fastness achieved by the vat dyes is significant, often giving a ratio ΔE(OXID)/ΔE(OXID+VAT) above 2.0, in some cases above 10.0.

The following table (scheme 11) describes the pyrazole primaries, conventional primaries, and couplers present in the Koleston Perfect color shades used in the above examples.

Scheme 11:

| | KP | KP | KP | KP | KP | KP | KP | KP |
|---|---|---|---|---|---|---|---|---|
| Koleston Perfect | 55/46 | 77/46 | 77/44 | 8/45 | 99/44 | 6/45 | 5/07 | 7/77 |
| pyrazole primaries | | | | | | | | |
| 4,5-diamino-1-hydroxyethylpyrazol | | • | • | • | • | | • | • |
| sulfate | | | | | | | | |
| 4,5-diamino-1-hexylpyrazol sulfate | • | | | • | | • | | |
| conventional primaries | | | | | | | | |
| 2-methoxymethyl-p- | • | | | | | • | • | • |
| phenylenediamine | | | | | | | | |
| 4-aminophenol | | | • | • | | • | | |
| couplers | | | | | | | | |
| resorcinol | | | | | • | | • | • |
| 4-amino-2-hydroxytoluene | • | | | | • | | • | |
| 2,4-diaminophenoxyethanol HCl | • | | | | | | | |
| 2-methyl-5- | • | | | | | | | |
| hydroxyethylaminophenol | | | | | | | | |
| 2-amino-4-hydroxyethyl- | | • | | | | • | | |
| aminoanisole sulfate, monohydrate | | | | | | | | |
| 2-methyl-5- | | • | • | • | | • | | • |
| hydroxyethylaminophenol | | | | | | | | |
| hydroxyethyl-3,4- | | | | | | | • | • |
| methylenedioxyaniline HCl | | | | | | | | |
| 2-methylresorcinol | | | | | | • | • | • |
| m-aminophenol | | | | | • | | | • |

The following statements further illustrate the present invention.
1 A method for coloring a keratinous substrate, comprising:
   a1 applying a composition A to said keratinous substrate, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof,
   a2 applying a composition B to said keratinous substrate, said composition B comprising an aqueous medium and a solubilized vat dye dissolved in said aqueous medium, b subsequent to step a2, applying a composition C to said keratinous substrate, said composition C comprising an oxidizing agent.
2 The method according to statement 1, wherein composition B is applied to said keratinous substrate simultaneously with or subsequent to applying composition A to said keratinous substrate.
3 The method according to statement 1 or 2, wherein compositions A and B are mixed prior to applying same to said keratinous substrate.
4 The method according to any of the preceding statements, wherein the oxidizing agent comprises hydrogen peroxide, in particular wherein the oxidizing agent is hydrogen peroxide in a concentration of from 1.0-12.0 vol %, based on the volume of composition C.
5 The method according to any of the preceding statements, wherein composition A has a pH in the range of 8.0-11.0, in particular in the range of 8.5-9.5, such as a pH of about 9.0.
6 The method according to any of the preceding statements, wherein composition B has a pH in the range of 8.0-11.0, in particular in the range of 8.5-9.5, such as a pH of about 9.0.
7 The method according to any of the preceding statements, wherein composition C has a pH in the range of 2.0-4.0, in particular in the range of 2.5.3.5.
8 The method according to any of the preceding statements, wherein prior to step b, compositions A and B are maintained on the keratinous substrate for at least 20 min, in particular 20-40 min, at a temperature of 20-40° C., in particular 35-40° C., and pH=8.0-11.0.
9 The method according to statement 8, further comprising prior to step b applying a temperature of 80-230° C. to the keratinous substrate, using a flat iron up to 30 seconds, in particular for 2-20 seconds.
10 The method according to any of the preceding statements, wherein during step b, composition C is maintained on the keratinous substrate for less than 30 min, in particular 10-20 min, at a temperature of 20-40° C., in particular 35-40° C., and pH=8.0-11.0.
11 The method according to any of the preceding statements, wherein during steps a and b, the pH is maintained in the range of from 8.0-10.0, for example in the range of from 8.5-9.5.
12 The method according to any of the preceding statements, wherein composition A, B, or both further comprise a penetration enhancer.
13 The method according to statement 12, wherein said penetration enhancer is selected from ammonia, urea, monoethanolamine, 2-amino-1-propanol, or a combination thereof.
14 The method according to any of the preceding statements, wherein composition A comprises a 4,5-diaminopyrazole according to Formula (1):

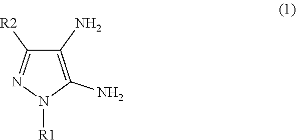

wherein R1 is linear or branched C1-C6 (hydroxy)alkyl and R2 is H or linear or branched C1-C6 (hydroxy)alkyl.
15 The method according to any of the preceding statements, wherein the 4,5-diaminopyrazole is selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.
16 The method according to any of the preceding statements, wherein composition A comprises an aminopyrazolopyridine according to Formula (2):

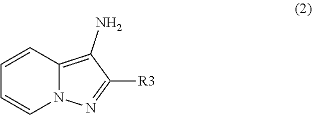

wherein R3 is (2-hydroxyethyl)oxy or 4,4-dimethylpiperazinium, or a combination thereof.
17 The method according to any of the preceding statements, wherein composition A comprises a ring-condensed 4,5-diaminopyrazolone according to Formula (3):

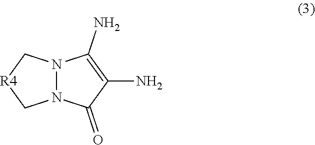

wherein R4 is methylene, ethylene, propylene, or a combination thereof.
18 The method according to any of the preceding statements, wherein composition A further comprises a conventional primary selected from N,N-bis(2-hydroxyethyl)-p-phenylenediamine, 4-aminophenol, p-phenylenediamine, 3-hydroxypropyl-p-phenylenediamine, 2-methyl-p-phenylenediamine, 2·methoxymethyl-p-phenylenediamine, or a combination thereof.
19 The method according to any of the preceding statements, wherein composition A comprises each primary in a concentration of 0.005-6.0 wt %, or 0.01-3.0 wt %, based on the weight of composition A.
20 The method according to any of the preceding statements, wherein composition A comprises a coupler selected from resorcinol, 2-methylresorcinol, hydroxyethyl-3,4-methylenedioxyaniline, m-aminophenol, 1-naphthol, 2-amino-4-hydroxyethylaminoanisole, 4-amino-2-hydroxytoluene, 2-methyl-5-hydroxyethylaminophenol, phenyl methyl pyrazolone, 6-hydroxy-benzomorpholine, 6-methoxy-2-methylamino-3-aminopyridine, or a combination thereof.
21 The method according to any of the preceding statements, wherein composition A comprises each coupler in a concentration of 0.005-6.0 wt % or 0.01-3.0 wt %, based on the weight of composition A.
22 The method according to any of the preceding statements, wherein composition B comprises two or more solubilized vat dyes.
23 The method according to any of the preceding statements, wherein the solubilized vat dye in composition B is a leuco vat dye sulfonic ester, or a combination of leuco vat dye sulfonic esters.
24 The method according to any of the preceding statements, wherein composition B comprises a solubilized vat dye selected from solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CAS #12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), or a combination thereof.
25 The method according to any of the preceding statements, wherein composition B comprises a solubilized vat dye comprising a polycyclic aromatic flatbed chromophore structure of four or more polycondensed rings optionally comprising one or more nitrogen atoms, said flatbed chromophore structure including a pyrene structural element according to Formula (4):

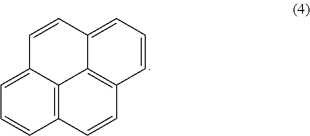 [SMILES: c1cc2ccc3cccc4ccc(c1)c2c34]

26 The method according to statement 25, wherein said solubilized vat dye comprises a polycyclic aromatic flatbed chromophore structure of at most eight polycondensed rings optionally comprising one or more nitrogen atoms, said flatbed chromophore structure including a pyranthrene structural element according to Formula (5):

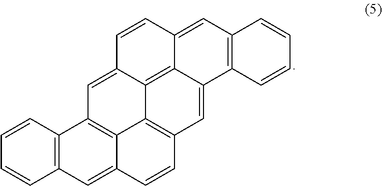 [SMILES: c1ccc2c(c1)cc1ccc3cc4c5ccccc5cc5ccc6cc2c1c3c6c54]

27 The method according to statement 25 or 26, wherein said solubilized vat dye comprises a polycyclic aromatic flatbed chromophore structure of six polycondensed rings optionally comprising one or more nitrogen atoms, said flatbed chromophore structure including a dibenzo-pyrene structural element according to Formula (6):

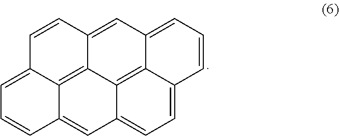 [SMILES: c1cc2ccc3cc4cccc5ccc6cc(c1)c2c3c6c54]

28 The method according to any of the preceding statements, wherein composition B comprises a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.
29 The method according to any of the preceding statements, wherein composition B comprises a reducing agent, in particular sodium sulfite, citric acid, ascorbic acid or a combination thereof.
30 The method according to any of the preceding statements, wherein composition B comprises each solubilized vat dye in a concentration of 0.50-2.0 wt %, based on the weight of composition B.
31 The method according to any of the preceding statements, wherein one or more of compositions A, B, C further comprises one or more components selected from pH adjusters, chelating agents, fragrances, care components, texture ingredients, medium reagents, solvents, surfactants.
32 The method according to any of the preceding statements, wherein composition A comprises at least 1.0 wt % of one or more anionic surfactants, at least 2.0 wt % of one or more linear or branched C14 to C30 fatty alcohols, and at least 0.1 wt % of one or more non-ionic surfactants.
33 The method according to any of statements 1-31, wherein composition A comprises at least 10 wt % of one or more hydrocarbons and at least 5.0 wt % of one or more fatty alcohols.
34 The method according to any of the preceding statements, wherein composition B comprises 10.0-30.0 wt % of one or more fatty acids.
35 The method according to statement 34, wherein composition B comprises 2.0-20.0 wt % of a rheology agent selected from ammonia, monoethanolamine, or a combination thereof.
36 The method according to any of statements 1-33, wherein composition B comprises 2.0-40.0 wt % anionic surfactant, 2.0-20.0 wt % amphoteric surfactant, and 0-10.0 wt % of an optional humectant.
37 The method according to any of the preceding statements, wherein one or more of compositions A, B, C is essentially free of pigments, parabens, silicones, or a combination thereof.
38 The method according to any of the preceding statements, wherein the color produced is in the color space range selected from orange, reddish-orange, copper, mahogany red, mahogany brownish, or vibrant red.
39 The method according to any of the preceding statements, wherein the wash fastness improvement ΔE(OXID)/ΔE(OXID+VAT) obtained is 1.5 or more, wherein:
   ΔE(OXID) is the color difference [(L2−L1)²+(a2−a1)²+(b2−b1)²]^{1/2},
   ΔE(OXID+VAT) is the color difference [(L4−L3)²+(a4−a3)²+(b4−b3)²]^{1/2},
   L1, a1 and b1 are the CIELAB L*a*b color coordinates, determined on natural white hair, colored with composition A alone, prior to washing,
   L2, a2 and b2 are the CIELAB L*a*b color coordinates, determined on natural white hair, colored with composition A alone, after 15 standard wash cycles,
   L3, a3 and b3 are the CIELAB L*a*b color coordinates, determined on natural white hair, colored with compositions A and B, prior to washing,
   L4, a4 and b4 are the CIELAB L.*a*b color coordinates, determined on natural white hair, colored with compositions A and B, after 15 standard wash cycles.
40 The method according to any of the preceding statements, further comprising rinsing the keratinous substrate subsequent to step b, optionally further comprising a post-treatment comprising shampooing, conditioning, drying the keratinous substrate, or a combination thereof.
41 The method according to any of the preceding statements, wherein the keratinous substrate is mammalian hair, in particular human hair.
42 A ready-to-use hair coloring formulation, said hair coloring formulation obtained by mixing a composition A with a composition 8, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, said composition B comprising an aqueous medium and a solubilized vat dye dissolved in said aqueous medium.
43 The formulation according to statement 42, having a pH in the range of 8.0-11.0, in particular in the range of 8.5-9.5, such as a pH of about 9.0.
44 The formulation according to statement 42 or 43, further comprising a penetration enhancer.
45 The formulation according to statement 44, wherein said penetration enhancer is selected from ammonia, urea, monoethanolamine, 2-amino-1-propanol, or a combination thereof.
46 The formulation according to any of statements 42-45, comprising a 4,5-diaminopyrazole according to Formula (1):

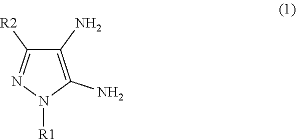

wherein R1 is linear or branched C1-C6 (hydroxy)alkyl and R2 is H or linear or branched C1-C6 (hydroxy)alkyl.
47 The formulation according to any of statements 42-46, wherein the 4,5-diaminopyrazole is selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.
48 The formulation according to any of statements 42-47, comprising an aminopyrazolopyridine according to Formula (2):

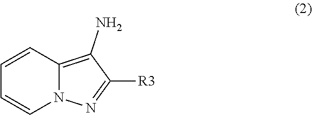

wherein R3 is (2-hydroxyethyl)oxy or 4,4-dimethylpiperazinium, or a combination thereof.
49 The formulation according to any of statements 42-48, comprising a ring-condensed 4,5-diaminopyrazolone according to Formula (3):

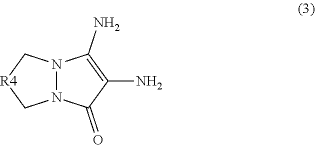

wherein R4 is methylene, ethylene, propylene, or a combination thereof.
50 The formulation according to any of statements 42-49, further comprising a conventional primary selected from N,N-bis(2-hydroxyethyl)-p-phenylenediamine, 4-aminophenol, p-phenylenediamine, 3-hydroxypropyl-p-phenylenediamine, 2-methyl-p-phenylenediamine, 2-methoxymethyl-p-phenylenediamine, or a combination thereof.
51 The formulation according to any of statements 42-50, comprising each primary in a concentration of 0.0025-3.0 wt %, or 0.005-1.5 wt %, based on the total weight of the formulation.
52 The formulation according to any of statements 42-51, comprising a coupler comprising resorcinol, 2-methylresorcinol, hydroxyethyl-3,4-methylenedioxyaniline, m-aminophenol, 1-naphthol, 2-amino-4-hydroxyethylaminoanisole, 4-amino-2-hydroxytoluene, 2-methyl-5-hydroxyethylaminophenol, phenyl methyl pyrazolone, 6-hydroxy-benzomorpholine, 6-methoxy-2-methylamino-3-aminopyridine, or a combination thereof.
53 The formulation according to any of statements 42-52, comprising each coupler in a concentration of 0.0025-3.0 wt %, or 0.005-1.5 wt %, based on the total weight of the formulation.
54 The formulation according to any of statements 42-53, comprising two or more solubilized vat dyes.
55 The formulation according to any of statements 42-54, wherein the solubilized vat dye is a leuco vat dye sulfonic ester, or a combination of leuco vat dye sulfonic esters.
56 The formulation according to any of statements 42-55, comprising a solubilized vat dye selected from solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CASI 12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), or a combination thereof.
57 The formulation according to any of statements 42-56, comprising a solubilized vat dye comprising a polycyclic aromatic flatbed chromophore structure of four or more polycondensed rings optionally comprising one or more nitrogen atoms, said flatbed chromophore structure including a pyrene structural element according to Formula (4):

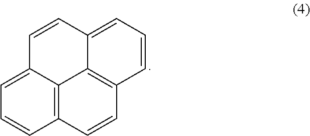 [SMILES: c1cc2ccc3cccc4ccc(c1)c2c34]

58 The formulation according to statement 57, wherein said solubilized vat dye comprises a polycyclic aromatic flatbed chromophore structure of at most eight polycondensed rings optionally comprising one or more nitrogen atoms, said flatbed chromophore structure including a pyranthrene structural element according to Formula (5):

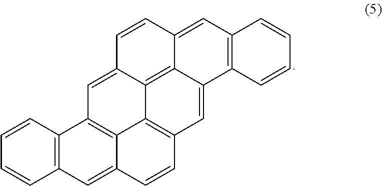 [SMILES: c1ccc2c(c1)cc1ccc3cc4c5ccccc5cc5ccc6cc2c1c3c6c54]

59 The formulation according to statement 57 or 58, wherein said solubilized vat dye comprises a polycyclic aromatic flatbed chromophore structure of six polycondensed rings optionally comprising one or more nitrogen atoms, said flatbed chromophore structure including a dibenzo-pyrene structural element according to Formula (6):

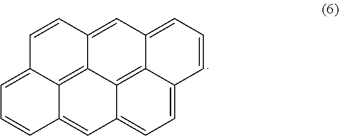 [SMILES: c1cc2ccc3cc4cccc5ccc6cc(c1)c2c3c6c54]

60 The formulation according to any of statements 42-59, comprising a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.
61 The formulation according to any of statements 42-60, comprising a reducing agent, in particular sodium sulfite, citric acid, ascorbic acid or a combination thereof.
62 The formulation according to any of statements 42-61, comprising each solubilized vat dye in a concentration of 0.25-1.0 wt %, based on the total weight of the formulation.
63 The formulation according to any of statements 42-62, further comprising one or more components selected from pH adjusters, chelating agents, fragrances, care components, texture ingredients, medium reagents, solvents, surfactants.
64 The formulation according to any of statements 42-63, wherein prior to mixing composition A comprises at least 1.0 wt % of one or more anionic surfactants, at least 2.0 wt % of one or more linear or branched C14 to C30 fatty alcohols, and at least 0.1 wt % of one or more non-ionic surfactants.
65 The formulation according to any of statements 42-63, wherein prior to mixing composition A comprises at least 10 wt % of one or more hydrocarbons and at least 5.0 wt % of one or more fatty alcohols.
66 The formulation according to any of statements 42-65, wherein prior to mixing composition B comprises 10.0-30.0 wt % of one or more fatty acids.
67 The formulation according to statement 66, wherein composition B comprises 2.0-20.0 wt % of a rheology agent selected from ammonia, monoethanolamine, or a combination thereof.
68 The formulation according to any of statements 42-65, wherein prior to mixing composition B comprises 2.0-40.0 wt % anionic surfactant, 2.0-20.0 wt % amphoteric surfactant, and 0-10.0 wt % of an optional humectant.
69 The formulation according to any of statements 42-68, wherein the formulation is essentially free of pigments, parabens, silicones, or a combination thereof.
70 The formulation according to any of statements 42-69, wherein the formulation is configured to produce a color is in the color space range selected from orange, reddish-orange, copper, mahogany red, mahogany brownish, or vibrant red.
71 The formulation according to any of statements 42-70, wherein the formulation is configured to provide a wash fastness improvement ΔE(OXID)/ΔE(OXID+VAT) of 1.5 or more, wherein:
   ΔE(OXID) is the color difference [(L2−L1)²+(a2−a1)²+(b2−b1)²]^{1/2},
   ΔE(OXID+VAT) is the color difference [(L4−L3)²+(a4−a3)²+(b4−b3)²]^{1/2},
   L1, a1 and b1 are the CIELAB L*a*b color coordinates, determined on natural white hair, colored with composition A alone, prior to washing,
   L2, a2 and b2 are the CIELAB L*a*b color coordinates, determined on natural white hair, colored with composition A alone, after 15 standard wash cycles,
   L3, a3 and b3 are the CIELAB L*a*b color coordinates, determined on natural white hair, colored with compositions A and B, prior to washing,
   L4, a4 and b4 are the CIELAB L*a*b color coordinates, determined on natural white hair, colored with compositions A and B, after 15 standard wash cycles.
72 A kit for coloring hair, comprising in separate compartments:
   a compartment A comprising a composition A, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, and
   a compartment B comprising a composition B, said composition B comprising an aqueous medium and a solubilized vat dye dissolved in said aqueous medium.
73 The kit according to statement 72, further comprising a compartment C comprising a composition C, said composition C comprising an oxidizing agent.
74 The kit according to statement 73, wherein the oxidizing agent comprises hydrogen peroxide, in particular wherein the oxidizing agent is hydrogen peroxide in a concentration of from 1.0-12.0 vol %, based on the volume of composition C.
75 The kit according to statement 73 or 74, wherein composition C has a pH in the range of 2.0-4.0, in particular in the range of 2.5-3.5.
76 The kit according to any of statements 72-75, wherein composition A has a pH in the range of 8.0-11.0, in particular in the range of 8.5-9.5, such as a pH of about 9.0.
77 The kit according to any of statements 72-76, wherein composition B has a pH in the range of 8.0-11.0, in particular in the range of 8.5-9.5, such as a pH of about 9.0.
78 The kit according to any of statements 72-77, wherein composition A, B, or both further comprise a penetration enhancer.
79 The kit according to statement 78, wherein said penetration enhancer is selected from ammonia, urea, monoethanolamine, 2-amino-1-propanol, or a combination thereof.
80 The kit according to any of statements 72-79, wherein composition A comprises a 4,5-diaminopyrazole is according to Formula (1):

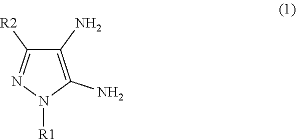

wherein R1 is linear or branched C1-C6 (hydroxy)alkyl and R2 is H or linear or branched C1-C6 (hydroxy)alkyl.
81 The kit according to any of statements 72-80, wherein the 4,5-diaminopyrazole is selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.
82 The kit according to any of statements 72-81, wherein composition A comprises an aminopyrazolopyridine according to Formula (2):

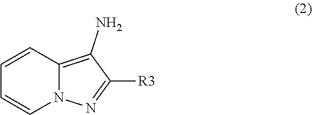

wherein R3 is (2-hydroxyethyl)oxy or 4,4-dimethylpiperazinium, or a combination thereof.
83 The kit according to any of statements 72-82, wherein composition A comprises a ring-condensed 4,5-diaminopyrazolone according to Formula (3):

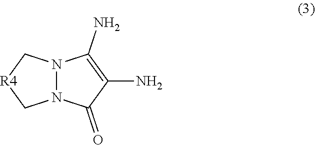

wherein R4 is methylene, ethylene, propylene, or a combination thereof.
84 The kit according to any of statements 72-83, wherein composition A further comprises a conventional primary selected from N,N-bis(2-hydroxyethyl)-p-phenylenediamine, 4-aminophenol, p-phenylenediamine, 3-hydroxypropyl-p-phenylenediamine, 2-methyl-p-phenylenediamine, 2-methoxymethyl-p-phenylenediamine, or a combination thereof.
85 The kit according to any of statements 72-84, wherein composition A comprises each primary in a concentration of 0.005-6.0 wt %, or 0.01-3.0 wt %, based on the weight of composition A.
86 The kit according to any of statements 72-85, wherein composition A comprises a coupler comprising resorcinol, 2-methylresorcinol, hydroxyethyl-3,4-methylenedioxyaniline, m-aminophenol, 1-naphthol, 2-amino-4-hydroxyethylaminoanisole, 4-amino-2-hydroxytoluene, 2-methyl-5-hydroxyethylaminophenol, phenyl methyl pyrazolone, 6-hydroxy-benzomorpholine, 6-methoxy-2-methylamino-3-aminopyridine, or a combination thereof.
87 The kit according to any of statements 72-86, wherein composition A comprises each coupler in a concentration of 0.005-6.0 wt %, or 0.01-3.0 wt %, based on the weight of composition A.
88 The kit according to any of statements 72-87, wherein composition B comprises two or more solubilized vat dyes.
89 The kit according to any of statements 72-88, wherein the solubilized vat dye in composition B is a leuco vat dye sulfonic ester, or a combination of leuco vat dye sulfonic esters.
90 The kit according to any of statements 72-89, wherein composition B comprises a solubilized vat dye selected from solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CAS #12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), or a combination thereof.
91 The kit according to any of statements 72-90, wherein composition B comprises a solubilized vat dye comprising a polycyclic aromatic flatbed chromophore structure of four or more polycondensed rings optionally comprising one or more nitrogen atoms, said flatbed chromophore structure including a pyrene structural element according to Formula (4):

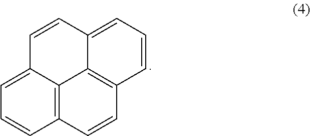 [SMILES: c1cc2ccc3cccc4ccc(c1)c2c34]

92 The kit according to statement 91, wherein said solubilized vat dye comprises a polycyclic aromatic flatbed chromophore structure of at most eight polycondensed rings optionally comprising one or more nitrogen atoms, said flatbed chromophore structure including a pyranthrene structural element according to Formula (5):

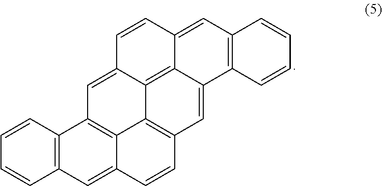 [SMILES: c1ccc2c(c1)cc1ccc3cc4c5ccccc5cc5ccc6cc2c1c3c6c54]

93 The kit according to statement 91 or 92, wherein said solubilized vat dye comprises a polycyclic aromatic flatbed chromophore structure of six polycondensed rings optionally comprising one or more nitrogen atoms, said flatbed chromophore structure including a dibenzo-pyrene structural element according to Formula (6):

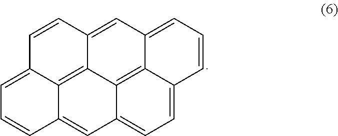 [SMILES: c1cc2ccc3cc4cccc5ccc6cc(c1)c2c3c6c54]

94 The kit according to any of statements 72-93, wherein composition B comprises a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.
95 The kit according to any of statements 72-94, wherein composition B comprises a reducing agent, in particular sodium sulfite, citric acid, ascorbic acid or a combination thereof.
96 The kit according to any of statements 72-95, wherein composition 8 comprises each solubilized vat dye in a concentration of 0.50-2.0 wt %, based on the weight of composition B.
97 The kit according to any of statements 72-96, wherein one or more of compositions A, B, C further comprises one or more components selected from pH adjusters, chelating agents, fragrances, care components, texture ingredients, medium reagents, solvents, surfactants.
98 The kit according to any of statements 72-97, wherein composition A comprises at least 1.0 wt % of one or more anionic surfactants, at least 2.0 wt % of one or more linear or branched C14 to C30 fatty alcohols, and at least 0.1 wt % of one or more non-ionic surfactants.
99 The kit according to any of statements 72-97, wherein composition A comprises at least 10 wt % of one or more hydrocarbons and at least 5.0 wt % of one or more fatty alcohols.
100 The kit according to any of statements 72-99, wherein composition B comprises 10.0-30.0 wt % of one or more fatty acids.
101 The kit according to statement 100, wherein composition B comprises 2.0-20.0 wt % of a rheology agent selected from ammonia, monoethanolamine, or a combination thereof.
102 The kit according to any of statements 72-99, wherein composition B comprises 2.0-40.0 wt % anionic surfactant, 2.0-20.0 wt % amphoteric surfactant, and 0-10.0 wt % of an optional humectant.
103 The kit according to any of statements 72-102, wherein one or more of compositions A, B, C is essentially free of pigments, parabens, silicones, or a combination thereof.
104 The kit according to any of statements 72-103, configured to produce a color is in the color space range selected from orange, reddish-orange, copper, mahogany red, mahogany brownish, or vibrant red.
105 The kit according to any of statements 72-104, configured to provide for a wash fastness improvement ΔE(OXID)/ΔE(OXID+VAT) obtained is 1.5 or more, wherein:
   ΔE(OXID) is the color difference [(L2−L1)²+(a2−a1)²+(b2−b1)²]^{1/2},
   · ΔE(OXID+VAT) is the color difference [(L4−L3)²+(a4−a3)²+(b4−b3)²]^{1/2},
   L1, a1 and b1 are the CIELAB L*a*b color coordinates, determined on natural white hair, colored with composition A alone, prior to washing,
   L2, a2 and b2 are the CIELAB L*a*b color coordinates, determined on natural white hair, colored with composition A alone, after 15 standard wash cycles,
   L3, a3 and b3 are the CIELAB L*a*b color coordinates, determined on natural white hair, colored with compositions A and B, prior to washing,
   L4, a4 and b4 are the CIELAB L*a*b color coordinates, determined on natural white hair, colored with compositions A and B, after 15 standard wash cycles.
106 A gel-like hair coloring component, comprising one or more solubilized vat dyes, each in a concentration of 0.50-2.0 wt %, 10.0-30.0 wt % of one or more saturated or unsaturated fatty acids, and 50-89.50 wt % water, wherein wt % each is based on the weight of the component.
107 The gel-like hair coloring component according to statement 106, comprising 12.0-25.0 wt %, in particular 15.0-20.0 wt % of said one or more saturated or unsaturated fatty acids.
108 The gel-like hair coloring component according to statement 106 or 107, wherein said one or more saturated or unsaturated fatty acids have a linear carbon chain length of C16-C30.
109 The gel-like hair coloring component according to any of statements 106-108, wherein said one or more saturated or unsaturated fatty acids comprise palmitic acid (C16:0), stearic acid (C18:0), palmitoleic acid (C16:1), oleic acid (C18:1), linoleic acid (C18:2), linolenic acid (C18:3), or a combination thereof.
110 The gel-like hair coloring component according to any of statements 106-109, comprising 2.0-20.0 wt % of a rheology agent selected from ammonia, monoethanolamine, or a combination thereof.
111 A shampoo-like hair coloring component, comprising one or more solubilized vat dyes, each in a concentration of 0.50-2.0 wt %, 2.0-40.0 wt % anionic surfactant, 2.0-20.0 wt % amphoteric surfactant, 0-10.0 wt % of an optional humectant, and 50.0-95.50 wt % water, wherein wt % each is based on the weight of the component.
112 The shampoo-like hair coloring component according to statement 111, comprising 4.0-25.0 wt %, in particular 6.0-15.0 wt % anionic surfactant, 3.0-15.0 wt %, in particular 4.0-10.0 wt % amphoteric surfactant, and 1.0-8.0 wt % of humectant.
113 The shampoo-like hair coloring component according to statement 111 or 112, wherein said anionic surfactant is selected from alkyl sulfates, alkyl ether sulfates, alkylamido ether sulphates, monoglyceride sulphates, a-olefin sulphonates, paraffin sulphonates, alkyl phosphates, alkyl sulphoacetates, acyl isethionates, alkyl ether phosphates, alkyl phenyl ether sulfonates, N-cocoyl fatty acyl glutamate, N-lauroyl glutamate, N-acylamino acid salts, N-coconut oil fatty acyl glycine, N-coconut oil fatty acyl alanine salts, salts of alkyl sulfosuccinic acids, and a combination thereof.
114 The shampoo-like hair coloring component according to any of statements 111-113, wherein said amphoteric surfactant is selected from betaines, in particular alkyl amido betaines, alkylbetaines, amidosulfobetaine, carboxybetaine-type betaines, sulfobetaine-type betaines, hydroxysulfobetaine betaines, phospobetaine-type betaines, and a combination thereof.
115 The shampoo-like hair coloring component according to any of statements 111-114, wherein said humectant is selected from glycerol, glycerol esters, propylene glycol, isopentyldiol, and a combination thereof.
116 The shampoo-like hair coloring component according to any of statements 111-115, comprising 6.0-10.0 wt % Sodium Laureth Sulfate, 4.0-6.0 wt % Cocoamidopropyl Betaine, and 2.0-4.0 wt % glycerin.
117 The hair coloring component according to any of statements 106-116, further comprising a penetration enhancer, in particular a penetration enhancer selected from ammonia, urea, monoethanolamine, 2-amino-1-propanol, or a combination thereof.
118 The hair coloring component according to any of statements 106-117, further comprising a reducing agent, in particular sodium sulfite, citric acid, ascorbic acid or a combination thereof.
119 The hair coloring component according to any of statements 106-118, further comprising one or more components selected from pH adjusters, chelating agents, fragrances, care components, texture ingredients, medium reagents, solvents, nonionic surfactants.
120 The hair coloring component according to any of statements 106-119, wherein the solubilized vat dye is a leuco vat dye sulfonic ester, or a combination of leuco vat dye sulfonic esters.
121 The hair coloring component according to any of statements 106-120, comprising a solubilized vat dye selected from solubilized vat violet 1 (CAS #1324-57-8), solubilized vat yellow 4 (CAS #3564-70-3), solubilized vat green 1 (CAS #2538-84-3), solubilized vat yellow 1 (CAS #6487 Sep. 8), solubilized vat red 34 (CAS #12226-70-9), solubilized vat red 10 (CAS #10126-90-6), solubilized vat orange 9 (CAS #70356 Jun. 8), solubilized vat orange 3 (CAS #10290 Mar. 6), solubilized vat yellow 7 (CAS #3956-62-5), or a combination thereof.
122 The hair coloring component according to any of statements 106-120, comprising a solubilized vat dye comprising a polycyclic aromatic flatbed chromophore structure of four or more polycondensed rings optionally comprising one or more nitrogen atoms, said flatbed chromophore structure including a pyrene structural element according to Formula (4):

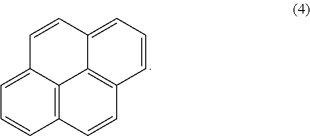 [SMILES: c1cc2ccc3cccc4ccc(c1)c2c34]

123 The hair coloring component according to statement 122, wherein said solubilized vat dye comprises a polycyclic aromatic flatbed chromophore structure of at most eight polycondensed rings optionally comprising one or more nitrogen atoms, said flatbed chromophore structure including a pyranthrene structural element according to Formula (5):

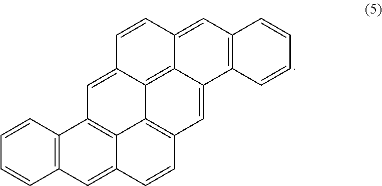 [SMILES: c1ccc2c(c1)cc1ccc3cc4c5ccccc5cc5ccc6cc2c1c3c6c54]

124 The hair coloring component according to statement 122 or 123, wherein said solubilized vat dye comprises a polycyclic aromatic flatbed chromophore structure of six polycondensed rings optionally comprising one or more nitrogen atoms, said flatbed chromophore structure including a dibenzo-pyrene structural element according to Formula (6):

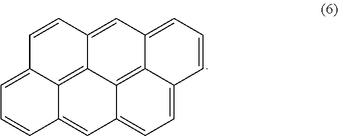 [SMILES: c1cc2ccc3cc4cccc5ccc6cc(c1)c2c3c6c54]

125 The hair coloring component according to any of statements 106-124, comprising a solubilized vat dye selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.
126 Use of a hair coloring component according to any of statements 106-125 for improving the wash fastness of hair color produced by oxidative dyes, wherein one or more of the oxidative dyes are derived from oxidative dye precursors comprising a pyrazole primary and a coupler.
127 The use according to statement 126, wherein one or more of the oxidative dyes are derived from oxidative dye precursors comprising a pyrazole primary comprising a 4,5-diaminopyrazole, an aminopyrazolopyridine, a ring-condensed 4,5-diaminopyrazolone, or a combination thereof.
128 The use according to statement 126 or 127, wherein one or more of the oxidative dyes are derived from oxidative dye precursors comprising a pyrazole primary comprising a 4,5-diaminopyrazole according to Formula (1):

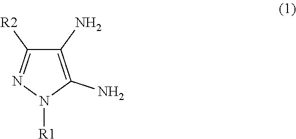

wherein R1 is linear or branched C1-C6 (hydroxy)alkyl and R2 is H or linear or branched C1-C6 (hydroxy)alkyl.
129 The use according to any of statements 126-128, wherein the 4,5-diaminopyrazole is selected from 4, S-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.
130 The use according to any statements 126-129, wherein one or more of the oxidative dyes are derived from oxidative dye precursors comprising a pyrazole primary comprising an aminopyrazolopyridine according to Formula (2):

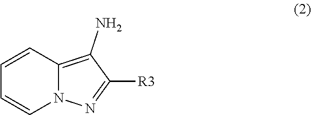

wherein R3 is (2-hydroxyethyl)oxy or 4,4-dimethylpiperazinium, or a combination thereof.
131 The use according to any of statements 126-130, wherein one or more of the oxidative dyes are derived from oxidative dye precursors comprising a pyrazole primary comprising a ring-condensed 4,5-diaminopyrazolone according to Formula (3):

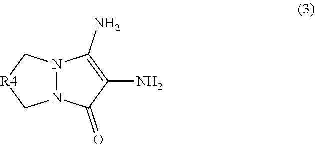

wherein R4 is methylene, ethylene, propylene, or a combination thereof.
132 The use according to any of statements 126-131, wherein the resulting hair color is in the orange, reddish-orange, copper, mahogany red, mahogany brownish, or vibrant red color space.
133 The use according to any of statements 126-132, wherein the wash fastness improvement
   ΔE(OXID)/ΔE(OXID+VAT) obtained is 1.5 or more, wherein:
   ΔE(OXID) is the color difference (L2−L1)²+(a2−a1)²+(b2−b1)²]^{1/2},
   ΔE(OXID+VAT) is the color difference [(L4−L3)²+(34−a3)+(b4−b3)²]^{1/2},
   L1, a1 and b1 are the CIELAB L*a*b color coordinates, determined on natural white hair, colored with composition A alone, prior to washing,
   0 L2, a2 and b2 are the CIELAB L*a*b color coordinates, determined on natural white hair, colored with composition A alone, after 15 standard wash cycles,
   L3, a3 and b3 are the CIELAB L*a*b color coordinates, determined on natural white hair, colored with compositions A and B, prior to washing,
   L4, a4 and b4 are the CIELAB L*a*b color coordinates, determined on natural white hair, colored with compositions A and B, after 15 standard wash cycles.
134 Use of a solubilized vat dye for improving the wash fastness of hair color produced by oxidative dyes, wherein one or more of the oxidative dyes are derived from oxidative dye precursors comprising a pyrazole primary and a coupler.
135 The use according to statement 134, wherein one or more of the oxidative dyes are derived from oxidative dye precursors comprising a pyrazole primary comprising a 4,5-diaminopyrazole, an aminopyrazolopyridine, a ring-condensed 4,5-diaminopyrazolone, or a combination thereof.
136 The use according to statement 134 or 135, wherein one or more of the oxidative dyes are derived from oxidative dye precursors comprising a pyrazole primary comprising 4,5-diaminopyrazole according to Formula (1):

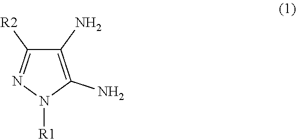

wherein R1 is linear or branched C1-C6 (hydroxy)alkyl and R2 is H or linear or branched C1-C6 (hydroxy)alkyl.
137 The use according to any of statements 134-136, wherein the 4,5-diaminopyrazole is selected from 4,5-diamino-1-methylpyrazol, 4,5-diamino-1-ethylpyrazol, 4,5-diamino-1-hexylpyrazol, 4,5-diamino-1-hydroxyethylpyrazol, 4,5-diamino-3-methylpyrazol, or a combination thereof.
138 The use according to any statements 134-137, wherein one or more of the oxidative dyes are derived from oxidative dye precursors comprising a pyrazole primary comprising an aminopyrazolopyridine according to Formula (2):

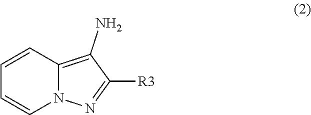

wherein R3 is (2-hydroxyethyl)oxy or 4,4-dimethylpiperazinium, or a combination thereof.
139 The use according to any of statements 134-138, wherein one or more of the oxidative dyes are derived from oxidative dye precursors comprising a pyrazole primary comprising a ring-condensed 4,5-diaminopyrazolone according to Formula (3):

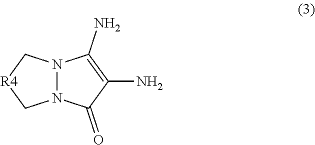

wherein R4 is methylene, ethylene, propylene, or a combination thereof.
140 The use according to any of statements 134-139, wherein the solubilized vat dye is a leuco vat dye sulfonic ester, or a combination of leuco vat dye sulfonic esters.
141 The use according to any of statements 134-140, wherein the solubilized vat dye comprises a polycyclic aromatic flatbed chromophore structure of four or more polycondensed rings optionally comprising one or more nitrogen atoms, said flatbed chromophore structure including a pyrene structural element according to Formula (4):

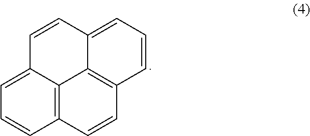 [SMILES: c1cc2ccc3cccc4ccc(c1)c2c34]

142 The use according to statement 141, wherein said solubilized vat dye comprises a polycyclic aromatic flatbed chromophore structure of at most eight polycondensed rings optionally comprising one or more nitrogen atoms, said flatbed chromophore structure including a pyranthrene structural element according to Formula (5):

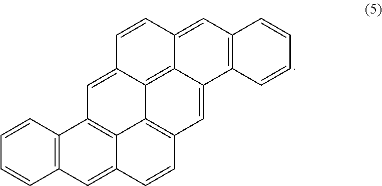 [SMILES: c1ccc2c(c1)cc1ccc3cc4c5ccccc5cc5ccc6cc2c1c3c6c54]

143 The use according to statement 141 or 142, wherein said solubilized vat dye comprises a polycyclic aromatic flatbed chromophore structure of six polycondensed rings optionally comprising one or more nitrogen atoms, said flatbed chromophore structure including a dibenzo-pyrene structural element according to Formula (6):

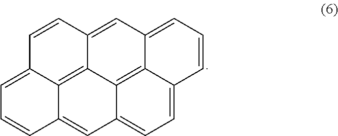 [SMILES: c1cc2ccc3cc4cccc5ccc6cc(c1)c2c3c6c54]

144 The use according to any of statements 134-143, wherein said solubilized vat dye is selected from solubilized vat yellow 4 (CAS #3564-70-3), solubilized vat yellow 1 (CAS #6487 Sep. 8), solubilized vat orange 3 (CAS #10290 Mar. 6), solubilized vat orange 9 (CAS #70356 Jun. 8), or a combination thereof.
145 The use according to any of statements 134-144, wherein the resulting hair color is in the orange, reddish-orange, copper, mahogany red, mahogany brownish, or vibrant red color space.
146 The use according to any of statements 134-145, wherein the wash fastness improvement ΔE(OXID)/ΔE(OXID+VAT) obtained is 1.5 or more, wherein:
   ΔE(OXID) is the color difference [(L2−L1)²+(a2−a1)²+(b2−b1)²]^{1/2},
   ΔE(OXID+VAT) is the color difference [(L4−L3)²+(a4−a3)²+(b4−b3)²]^{1/2},
   L1, a1 and b1 are the CIELAB L*a*b color coordinates, determined on natural white hair, colored with composition A alone, prior to washing,
   L2, a2 and b2 are the CIELAB L*a*b color coordinates, determined on natural white hair, colored with composition A alone, after 15 standard wash cycles,
   L3, a3 and b3 are the CIELAB L*a*b color coordinates, determined on natural white hair, colored with compositions A and B, prior to washing,
   L4, a4 and b4 are the CIELAB L*a*b color coordinates, determined on natural white hair, colored with compositions A and B, after 15 standard wash cycles.

1. A method for coloring a keratinous substrate, comprising:
a1 applying a composition A to said keratinous substrate, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof,
a2 applying a composition B to said keratinous substrate, said composition B comprising an aqueous medium and a solubilized vat dye dissolved in said aqueous medium, wherein the solubilized vat dye is a leuco vat dye sulfonic ester,
b subsequent to step a2, applying a composition C to said keratinous substrate, said composition C comprising an oxidizing agent.

2. The method according to claim 1, wherein composition B is applied to said keratinous substrate simultaneously with or subsequent to applying composition A to said keratinous substrate or wherein compositions A and B are mixed prior to applying same to said keratinous substrate.

3. (canceled)

4. (canceled)

5. The method according to claim 1, wherein composition A, B, or both, has a pH in the range of 8.0-11.0, in particular in the range of 8.5-9.5, such as a pH of about 9.0.

6. (canceled)

7. (canceled)

8. The method according to claim 1, wherein composition A, B, or both further comprise a penetration enhancer, in particular a penetration enhancer selected from ammonia, urea, monoethanolamine, 2-amino-1-propanol, or a combination thereof.

9. (canceled)

10. (canceled)

11. (canceled)

12. (canceled)

13. (canceled)

14. (canceled)

15. (canceled)

16. (canceled)

17. (canceled)

18. (canceled)

19. The method according to claim 1, wherein composition B comprises a reducing agent, in particular sodium sulfite, citric acid, ascorbic acid or a combination thereof.

20. (canceled)

21. (canceled)

22. (canceled)

23. The method according to claim 1, wherein the keratinous substrate is mammalian hair, in particular human hair.

24. A ready-to-use hair coloring formulation, said hair coloring formulation obtained by mixing a composition A with a composition B, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, said composition B comprising an aqueous medium and a solubilized vat dye dissolved in said aqueous medium, wherein the solubilized vat dye is a leuco vat dye sulfonic ester.

25. The formulation according to claim 24, having a pH in the range of 8.0-11.0, in particular in the range of 8.5-9.5, such as a pH of about 9.0.

26. The formulation according to claim 24, further comprising a penetration enhancer, in particular a penetration enhancer selected from ammonia, urea, monoethanolamine, 2-amino-1-propanol, or a combination thereof.

27. (canceled)

28. (canceled)

29. (canceled)

30. (canceled)

31. (canceled)

32. (canceled)

33. (canceled)

34. (canceled)

35. (canceled)

36. (canceled)

37. The formulation according to claim 24, comprising a reducing agent, in particular sodium sulfite, citric acid, ascorbic acid or a combination thereof.

38. (canceled)

39. (canceled)

40. A kit for coloring hair, comprising in separate compartments:
a compartment A comprising a composition A, said composition A comprising an aqueous medium, and oxidative dye precursors dissolved in said aqueous medium, said oxidative dye precursors comprising a pyrazole primary and a coupler, wherein the pyrazole primary comprises a 4,5-diaminopyrazole, an aminopyrazolopyridine, a ring-condensed 4,5-diaminopyrazolone, or a combination thereof, and
a compartment B comprising a composition B, said composition B comprising an aqueous medium and a solubilized vat dye dissolved in said aqueous medium, wherein the solubilized vat dye is a leuco vat dye sulfonic ester.

41. The kit according to claim 40, further comprising a compartment C comprising a composition C, said composition C comprising an oxidizing agent.

42. (canceled)

43. The kit according to claim 41, wherein composition C has a pH in the range of 2.0-4.0, in particular in the range of 2.5-3.5.

44. The kit according to claim 40, wherein composition A, composition B, or both, have a pH in the range of 8.0-11.0, in particular in the range of 8.5-9.5, such as a pH of about 9.0.

45. The kit according to claim 40, wherein composition A, B, or both, further comprise a penetration enhancer, in particular a penetration enhancer selected from ammonia, urea, monoethanolamine, 2-amino-1-propanol, or a combination thereof.

46. (canceled)

47. (canceled)

48. (canceled)

49. (canceled)

50. The kit according to claim 40, wherein composition A further comprises a conventional primary selected from N,N-bis(2-hydroxyethyl)-p-phenylenediamine, 4-aminophenol, p-phenylenediamine, 3-hydroxypropyl-p-phenylenediamine, 2-methyl-p-phenylenediamine, 2-methoxymethyl-p-phenylenediamine, or a combination thereof.

51. (canceled)

52. (canceled)

53. (canceled)

54. (canceled)

55. (canceled)

56. The kit according to claim 40, wherein composition B comprises a reducing agent, in particular sodium sulfite, citric acid, ascorbic acid or a combination thereof.

57. (canceled)

58. (canceled)

59. A gel-like hair coloring component, comprising one or more leuco vat dye sulfonic esters, each in a concentration of 0.50-2.0 wt %, 10.0-30.0 wt % of one or more saturated or unsaturated fatty acids, and 50-89.50 wt % water, wherein wt % each is based on the weight of the component.

60. (canceled)

61. A shampoo-like hair coloring component, comprising one or more leuco vat dye sulfonic esters, each in a concentration of 0.50-2.0 wt %, 2.0-40.0 wt % anionic surfactant, 2.0-20.0 wt % amphoteric surfactant, 0-10.0 wt % of an optional humectant, and 50.0-95.50 wt % water, wherein wt % each is based on the weight of the component.

62. (canceled)

63. (canceled)

64. (canceled)

65. (canceled)

66. A method of improving the wash fastness of hair color produced by oxidative dyes, the method comprising applying gone or more leuco vat dye sulfonic esters, or the gel-like hair coloring component according to claim 18, or the shampoo-like hair coloring component according to claim 19, to hair colored with said oxidative dyes, wherein one or more of said oxidative dyes are derived from oxidative dye precursors comprising a pyrazole primary and a coupler, optionally wherein one or more of said oxidative dyes are derived from oxidative dye processors comprising a pyrazole primary comprising a 4,5-diaminopyrazole an aminopyrazolopyridine, a ring-condensed 4,5-diaminopyrazolone, or a combination thereof.

67. (canceled)

68. (canceled)

69. (canceled)

70. (canceled)

71. (canceled)

72. (canceled)

73. (canceled)

74. (canceled)

75. (canceled)

76. (canceled)

77. (canceled)
